(19) United States
(12) Patent Application Publication
(10) Pub. No.: US 2021/0389321 A1
(43) Pub. Date: Dec. 16, 2021

(54) SYSTEMS AND METHODS FOR ANALYTE DETECTION USING ELECTROMAGNETICALLY INDUCED RESONANCE

(71) Applicants: Massachusetts Institute of Technology, Cambridge, MA (US); The Brigham and Women's Hospital, Inc., Boston, MA (US)

(72) Inventors: Sumanth Kaushik, Belmont, MA (US); Jeffrey Karp, Brookline, MA (US); Charles Wynn, Groton, MA (US); Anthony Samir, Newton, MA (US)

(21) Appl. No.: 17/339,889

(22) Filed: Jun. 4, 2021

Related U.S. Application Data
(60) Provisional application No. 63/034,813, filed on Jun. 4, 2020.

(51) Int. Cl. G01N 33/569; G01N 22/00; G01N 21/64

(52) U.S. Cl. CPC G01N 33/56983; G01N 22/00; G01N 2333/165; G01N 2201/06113; G01N 2201/068; G01N 21/64

(57) ABSTRACT

A system and method is provided for detecting an analyte within a sample. The method includes providing a first electromagnetic radiation to the sample in a manner that resonantly excites mechanical vibrations in the analyte. The method also includes providing a second electromagnetic radiation to the sample so as to interact with vibrating analyte, wherein a third electromagnetic radiation is produced based on the interaction. The method further includes receiving the a third electromagnetic radiation and determining the presence of the analyte based on the received a third electromagnetic radiation.

### CROSS-REFERENCE TO RELATED APPLICATIONS

This application claims priority to U.S. Patent Application No. 63/034,813 filed Jun. 4, 2020, and entitled, “Systems and Methods for Analyte Detection using Electromagnetically Induced Resonance,” which is hereby incorporated by reference in its entirety.

### STATEMENT REGARDING FEDERALLY SPONSORED RESEARCH

This invention was made with government support under FA8702-15-D-0001 awarded by the United States Air Force. The government has certain rights in the invention.

### BACKGROUND

The COVID-19 (also referred to as simply “coronavirus”) pandemic caused by severe acute respiratory syndrome coronavirus 2 (“SARS-CoV-2”) has exposed various limitations of previously available testing equipment and methods within the medical field. A lack of testing has been proposed as a leading reason for the ongoing case growth of COVID-19 in the United States and other countries. A higher level of testing is necessary to identify the infected individuals and to isolate them. Presently with COVID-19, individuals with positive tests are estimated to represent only a small fraction of all infected, with the proportion of undetected cases estimated to be as high as 86%. Moreover, asymptomatic cases are suspected to play a substantial role in transmission of COVID-19, implying that testing must be deployed into the very large asymptomatic population if testing is to be maximally effective in preventing disease spread. This highlights the utmost importance of developing a sensitive, specific, rapid, simple-to deploy, and supply chain robust testing method for COVID-19.

The United States has issued Emergency Use Authorization for SARS-CoV-2 diagnosis, allowing tests to be offered based on manufacturer-reported data without formal FDA clearance. In response, numerous nucleic acid and serological tests have been developed and marketed. As of Can 7, 2020, there are 388 tests, of which only 58 have been approved by the FDA. Whether a specific test can be used at large-scale setting depends on the assay time, throughput, limit of detection, accuracy, costs, and production limits. The ability of a test to identify active infection is often prioritized as this has the greatest potential impact on limiting transmission. Accordingly, various viral RNA and antigen detection methods are being used. However, many of these tests are time consuming and limited by reagent availability. One the fastest current polymerase chain reaction (“PCR”) techniques is nicking endonuclease amplification reaction (“NEAR”), which takes 15 minutes to produce results, but unfortunately is a low throughput test and not optimal for large-scale population testing, which requires fast, high throughput and inexpensive testing. The various RNA and antigen testing solutions all suffer from the same need for reagents.

Thus, what is needed are novel systems and methods capable of successfully detecting the COVID-19 virus in a subject. Furthermore, in order to more successfully address future pandemics or assist in the detection of microorganisms, a general assay method of identifying one or more pathogens that improves on prior attempts is highly desirable.

### SUMMARY OF THE DISCLOSURE

The present disclosure addresses the aforementioned drawbacks by providing novel systems and methods for scalable, reagent-free, supply chain robust, simple, and widely deployable detecting of pathogens, nanostructures, and other analytes.

In one aspect of the present disclosure, a method is provided for detecting an analyte within a sample. The method can include providing at least one first electromagnetic radiation to the sample in a manner that resonantly excites mechanical vibrations in the analyte. The method can also include providing at least one second electromagnetic radiation to the sample so as to interact with vibrating analyte, wherein at least one third electromagnetic radiation can be produced based on the interaction. The method can further include receiving the at least one third electromagnetic radiation and determining the presence of the analyte based on the received at least one third electromagnetic radiation.

In another aspect of the present disclosure, a system is provided for detecting an analyte within a sample. The system can include a first energy source configured to provide at least one first electromagnetic radiation to the sample in a manner that resonantly excites mechanical vibrations in the analyte and a second energy source configured to provide at least one second electromagnetic radiation to the sample so as to interact with vibrating analyte. The at least one third electromagnetic radiation can be produced based on the interaction. The system can further include a receiver including a filter configured to separate the at least one third electromagnetic radiation from background electromagnetic radiation and a detector configured to receive the filtered at least one third electromagnetic radiation and produce at least one detection signal. The system can also include a processor configured to provide information regarding the presence of the analyte based on the detection signal.

In one aspect of the present disclosure, a method is provided for detecting multiple target pathogens in a subject. The method can include obtaining a sample from the subject and providing at least one first electromagnetic radiation to the sample in a manner that resonantly excites mechanical vibrations in the target pathogens. The frequency of the first electromagnetic radiation can be swept over a target frequency range in which resonant frequencies of the target pathogens exist. The method can further include providing at least one second electromagnetic radiation to the sample so as to interact with vibrating target pathogens, such that multiple scattered electromagnetic radiation are produced based on the interaction. The method can also include receiving the multiple scattered electromagnetic radiation and determining the presence of the target pathogens based on the received multiple scattered electromagnetic radiation.

In another aspect the present disclosure, a method is provided for detecting SARS-CoV-2 within a subject. The method can include obtaining a sample from the subject, providing at least one first electromagnetic radiation to the sample in a manner that resonantly excites mechanical vibrations for at least one SARS-CoV-2 particle, and providing at least one second electromagnetic radiation to the sample so as to interact with the vibrating SARS-CoV-2 particle, where the at least one third electromagnetic radiation is produced based on the interaction. The method can further include receiving the at least one third electromagnetic radiation and determining the presence of SARS-CoV-2 within the subject based on the received at least one third electromagnetic radiation.

In yet another aspect of the present disclosure, a method of detecting a pathogen within a sample is provided that can include exciting the sample with a first electromagnetic radiation selected to cause mechanically resonant vibrations in the pathogen. The method can also include exciting the sample with a second electromagnetic radiation having a selected frequency configured to be Doppler shifted upon interacting with the mechanically resonant vibrations of pathogen to thereby create a Doppler-shifted electromagnetic radiation and acquiring a sample of electromagnetic radiation from the sample that includes at least the second electromagnetic radiation and the Doppler-shifted electromagnetic radiation. The method can further include filtering the sample of the electromagnetic radiation from the sample to remove the second electromagnetic radiation, analyzing the filtered sample of the electromagnetic radiation to determine a presence of the Doppler-shifted electromagnetic radiation, and, upon determining the Doppler-shifted electromagnetic radiation, indicating the presence of the pathogen in the sample.

Some non-limiting examples of the disclosure provide a method of detecting an analyte within a sample. The method can include providing a first electromagnetic radiation to the sample in a manner that resonantly excites mechanical vibrations in the analyte, and providing a second electromagnetic radiation to the sample so as to interact with the vibrating analyte. A third electromagnetic radiation can be produced based on the interaction. The method can include receiving the third electromagnetic radiation and determining the presence of the analyte based on the received a third electromagnetic radiation.

In some non-limiting examples, the first electromagnetic radiation has a substantially uniform frequency equal to a resonance frequency of the analyte.

In some non-limiting examples, the first electromagnetic radiation is within the microwave range.

In some non-limiting examples, the frequency of the first electromagnetic radiation is swept over a target frequency range in which a resonant frequency of the analyte exists.

In some non-limiting examples, the frequency of the first electromagnetic radiation is swept at a rate of 1 GHz per second over the target frequency range.

In some non-limiting examples, the first electromagnetic radiation has a frequency of more than 1 GHz, more than 3 GHz, more than 5 GHz, more than 8 GHz, less than 100 GHz, less than 50 GHz, less than 15 GHz, between 1 GHz and 100 GHz, between 3 GHz and 50 GHz, or between 5 GHz and 15 GHz.

In some non-limiting examples, the third electromagnetic radiation is produced by the second electromagnetic radiation scattering off of the vibrating analyte and undergoing a phase modulation and a spectrum change.

In some non-limiting examples, the frequency of the third electromagnetic radiation is shifted from the second electromagnetic radiation by an amount equal to the resonance frequency of the analyte.

In some non-limiting examples, the method step of receiving the third electromagnetic radiation includes measuring the spectrum of the third electromagnetic radiation.

In some non-limiting examples, the method step of receiving the third electromagnetic radiation comprises filtering out the third electromagnetic radiation from background electromagnetic radiation.

In some non-limiting examples, the third electromagnetic radiation is passed through a reference cell with an absorption line matched to the frequency of the second electromagnetic radiation.

In some non-limiting examples, the reference cell is selected from the group consisting of a cesium vapor cell, a potassium vapor cell, a sodium vapor cell, a rubidium vapor cell, and an iodine vapor cell.

In some non-limiting examples, the reference cell is a rubidium 780 nm vapor cell. The second electromagnetic radiation can have a substantially uniform wavelength of 780 nm.

In some non-limiting examples, the second electromagnetic radiation is within the visible light range or the infrared range.

In some non-limiting examples, the method can include determining a concentration of the analyte within the sample based on the third electromagnetic radiation.

In some non-limiting examples, at least a part of the method steps of providing the first electromagnetic radiation and providing the second electromagnetic radiation can occur simultaneously.

In some non-limiting examples, the method steps of providing the first electromagnetic radiation, providing the second electromagnetic radiation, and receiving the third electromagnetic radiation can all occur within a time period of less than 15 seconds.

In some non-limiting examples, the first electromagnetic radiation is provided to the sample in an orthogonal direction to the second electromagnetic radiation.

In some non-limiting examples, the method step of determining the presence of the analyte includes comparing the spectrum of the received a third electromagnetic radiation to previously acquired spectra of a reference sample containing the analyte.

In some non-limiting examples, the analyte is a pathogen.

In some non-limiting examples, the pathogen is SARS-CoV-2.

In some non-limiting examples, the method can include upon determining the presence of the analyte, providing a control signal indicating the presence of the analyte.

In some non-limiting examples, the method can include triggering operation of a system configured to control or reduce the presence of the analyte based on the control signal.

In some non-limiting examples, the system configured to control or reduce the presence of the analyte includes at least one of a heating, ventilation, and air conditioning (HVAC) system, an ultraviolet (UV) disinfectant system, or a filtering system.

In some non-limiting examples, one of the second electromagnetic radiation includes a lock-in signal phased based on the first electromagnetic radiation or a fourth electromagnetic radiation configured to serve as a lock-in reference signal.

Some non-limiting examples of the disclosure provide a system for detecting an analyte within a sample. The system can include a first energy source configured to provide a first electromagnetic radiation to the sample in a manner that resonantly excites mechanical vibrations in the analyte, and a second energy source configured to provide a second electromagnetic radiation to the sample so as to interact with vibrating analyte. A third electromagnetic radiation can be produced based on the interaction. The system can include a receiver. The receiver can include a filter configured to separate the third electromagnetic radiation from background electromagnetic radiation, and a detector configured to receive the filtered a third electromagnetic radiation and produce a detection signal. The system can include a processor configured to provide information regarding the presence of the analyte based on the detection signal.

In some non-limiting examples, the first electromagnetic radiation has a substantially uniform frequency equal to a resonance frequency of the analyte.

In some non-limiting examples, the first energy source is a microwave emitter and the first electromagnetic radiation is within the microwave range.

In some non-limiting examples, the system can include a microwave receiver positioned on the opposite side of the sample from the first energy source and configured to receive a portion of the first electromagnetic radiation and provide a feedback signal to the first energy source, the processor, or both.

In some non-limiting examples, the first energy source is configured to sweep the frequency of the first electromagnetic radiation over a target frequency range in which a resonant frequency of the analyte exists.

In some non-limiting examples, the frequency of the first electromagnetic radiation is swept at a rate of 1 GHz per second over the target frequency range.

In some non-limiting examples, the first electromagnetic radiation has a frequency of more than 1 GHz, more than 3 GHz, more than 5 GHz, more than 8 GHz, less than 100 GHz, less than 50 GHz, less than 15 GHz, between 1 GHz and 100 GHz, between 3 GHz and 50 GHz, or between 5 GHz and 15 GHz.

In some non-limiting examples, the second energy source can be a laser emitter and the second electromagnetic radiation can be within the visible light range or the infrared range.

In some non-limiting examples, the system can include a sample holder configured to maintain a constant position of a portion of the sample. The portion of the sample can be within the pathways of the first electromagnetic radiation and the second electromagnetic radiation.

In some non-limiting examples, the sample holder is formed of a material configured to produce a Brillouin signal having a frequency offset from the third electromagnetic radiation.

In some non-limiting examples, the detection signal includes information on the spectrum of the third electromagnetic radiation.

In some non-limiting examples, the filter includes a reference cell with an absorption line matched to the frequency of the second electromagnetic radiation.

In some non-limiting examples, the reference cell is selected from the group consisting of a cesium vapor cell, a potassium vapor cell, a sodium vapor cell, a rubidium vapor cell, and an iodine vapor cell.

In some non-limiting examples, the reference cell is a rubidium 780 nm vapor cell. The second electromagnetic radiation can have a substantially uniform wavelength of 780 nm.

In some non-limiting examples, the receiver is positioned on the same side of the sample as the second energy source so as to receive scattered a second electromagnetic radiation.

In some non-limiting examples, the system can include a second receiver. The second receiver can include a second filter configured to separate the third electromagnetic radiation from background electromagnetic radiation, and a second detector configured to receive the filtered third electromagnetic radiation and produce a detection signal. The second receiver can be positioned on the opposite side of the sample as the second energy source so as to receive transmitted a second electromagnetic radiation.

In some non-limiting examples, the system can include a display in electrical communication with the processor and can be configured to display information regarding the presence of the analyte.

In some non-limiting examples, the system can include an interface in electrical communication with the first energy source, the second energy source, the receiver, and the processor. The interface can be configured to allow a user to control operating parameters of the first energy source or the second energy, adjust processing parameters of the processor, or both.

In some non-limiting examples, the processor is configured to determine a concentration of the analyte within the sample based on the third electromagnetic radiation.

In some non-limiting examples, the first energy source and the second energy source are arranged to provide the first electromagnetic radiation and the second electromagnetic radiation sample in an orthogonal direction to the sample.

In some non-limiting examples, the processor is configured to compare the spectrum of the received a third electromagnetic radiation to previously acquired spectra of a reference sample containing the analyte in order to determine the information regarding the presence of the analyte.

Some non-limiting examples of the disclosure provide a method of detecting multiple target pathogens in a subject. The method can include obtaining a sample from the subject, and providing a first electromagnetic radiation to the sample in a manner that resonantly excites mechanical vibrations in the target pathogens. The frequency of the first electromagnetic radiation can be swept over a target frequency range in which resonant frequencies of the target pathogens exist. The method can include providing a second electromagnetic radiation to the sample so as to interact with vibrating target pathogens. Multiple scattered electromagnetic radiation can be produced based on the interaction. The method can include receiving the multiple scattered electromagnetic radiation, and determining the presence of the target pathogens based on the received multiple scattered electromagnetic radiation.

Some non-limiting examples of the disclosure provide a method of detecting SARS-CoV-2 within a subject. The method can include obtaining a sample from the subject, providing a first electromagnetic radiation to the sample in a manner that resonantly excites mechanical vibrations a SARS-CoV-2 particle, and providing a second electromagnetic radiation to the sample so as to interact with vibrating SARS-CoV-2 particle. A third electromagnetic radiation can be produced based on the interaction. The method can include receiving the third electromagnetic radiation; and determining the presence of SARS-CoV-2 within the subject based on the received a third electromagnetic radiation.

In some non-limiting examples, the sample is selected from the group consisting of saliva, sputum, blood, breath exhalant, a nasopharyngeal swab, an oropharyngeal swab, stool, and a surface swab.

In some non-limiting examples, the subject is a human.

Some non-limiting examples of the disclosure provide a method of detecting a pathogen within a sample. The method can include exciting the sample with a first electromagnetic radiation selected to cause mechanically resonant vibrations in the pathogen, exciting the sample with a second electromagnetic radiation having a selected frequency configured to be Doppler shifted upon interacting with the mechanically resonant vibrations of pathogen to thereby create a Doppler-shifted electromagnetic radiation, and acquiring a sample of electromagnetic radiation from the sample that includes at least the second electromagnetic radiation and the Doppler-shifted electromagnetic radiation. The method can include filtering the sample of the electromagnetic radiation from the sample to remove the second electromagnetic radiation, analyzing the filtered sample of the electromagnetic radiation to determine a presence of the Doppler-shifted electromagnetic radiation, and upon determining the Doppler-shifted electromagnetic radiation, indicating the presence of the pathogen in the sample.

Some non-limiting examples of the disclosure provide a method of detecting SARS-CoV-2 within a subject. The method can include obtaining a sample from the subject, providing a first electromagnetic radiation to the sample in a manner that resonantly excites mechanical vibrations a SARS-CoV-2 particle, and providing a second electromagnetic radiation to the sample so as to interact with vibrating SARS-CoV-2 particle. A third electromagnetic radiation can be produced based on the interaction. The method can include receiving the a third electromagnetic radiation, and determining the presence of SARS-CoV-2 within the subject based on the received a third electromagnetic radiation.

In some non-limiting examples, the sample is selected from the group consisting of saliva, sputum, blood, breath exhalant, a nasopharyngeal swab, an oropharyngeal swab, stool, and a surface swab.

In some non-limiting examples, the subject is a human.

Some non-limiting examples of the disclosure provide a method of detecting a pathogen within a sample. The method can include exciting the sample with a first electromagnetic radiation selected to cause mechanically resonant vibrations in the pathogen, and exciting the sample with a second electromagnetic radiation having a selected frequency configured to be Doppler shifted upon interacting with the mechanically resonant vibrations of pathogen to thereby create a Doppler-shifted electromagnetic radiation, The method can include acquiring a sample of electromagnetic radiation from the sample that includes at least the second electromagnetic radiation and the Doppler-shifted electromagnetic radiation, filtering the sample of the electromagnetic radiation from the sample to remove the second electromagnetic radiation;, analyzing the filtered sample of the electromagnetic radiation to determine a presence of the Doppler-shifted electromagnetic radiation, and upon determining the Doppler-shifted electromagnetic radiation, indicating the presence of the pathogen in the sample.

Some non-limiting examples of the disclosure provide a computer-implemented method for determining a resonant frequency of an analyte. The method can include causing, using one or more computing devices, a first energy source to emit first electromagnetic radiation towards a sample that includes the analyte, causing, using the one or more computing devices, a second energy source to emit second electromagnetic radiation, different from the first electromagnetic radiation, towards the sample that includes the analyte, receiving, using a detector and the one or more computing devices, third electromagnetic radiation different from the first electromagnetic radiation and the second electromagnetic radiation, and determining, using the one or more computing devices, a resonant frequency of the analyte based on the received third electromagnetic radiation.

In some non-limiting examples, the method can include generating, using the one or more computing devices, a spectrum based on the received third electromagnetic radiation, determining, using the one or more computing devices, a peak within the spectrum, and determining, using the one or more computing devices, the resonant frequency of the analyte based on the peak within the spectrum.

In some non-limiting examples, the method can include determining, using the one or more computing devices, a plurality of peaks within the spectrum, and determining, using the one or more computing devices, the resonant frequency of the analyte based on the plurality of peaks.

Some non-limiting examples of the disclosure provide a computer-implemented method for detecting an analyte. The method can include causing, using one or more computing devices, a first energy source to emit first electromagnetic radiation towards a sample that includes the analyte, causing, using the one or more computing devices, a second energy source to emit second electromagnetic radiation, different from the first electromagnetic radiation, towards the sample that includes the analyte, receiving, using a detector and the one or more computing devices, third electromagnetic radiation different from the first electromagnetic radiation and the second electromagnetic radiation, and determining, using the one or more computing devices, a presence or concentration of the analyte based on the received third electromagnetic radiation.

In some non-limiting examples, the analyte is a virus.

In some non-limiting examples, determining the presence or concentration of the analyte does not include the analyte binding to a detector compound.

In some non-limiting examples, the detector compound includes an antibody that binds to the analyte.

In some non-limiting examples, determining the presence or concentration of the analyte can include generating, using the one or more computing devices, a spectrum based on the received third electromagnetic radiation, determining, using the one or more computing devices, a peak within the spectrum, and determining, using the one or more computing devices, the presence or concentration of the analyte based on the peak within the spectrum.

In some non-limiting examples, the sample includes material from multiple different sources.

Some non-limiting examples of the disclosure provide a method of detecting an analyte within a sample. The method can include providing a first electromagnetic radiation to the sample in a manner that resonantly excites mechanical vibrations in the analyte, providing a second electromagnetic radiation to the sample so as to interact with the vibrating analyte, wherein a third electromagnetic radiation is produced based on the interaction, receiving the third electromagnetic radiation, and determining the presence of the analyte based on the received a third electromagnetic radiation.

In some non-limiting examples, the first electromagnetic radiation has a substantially uniform frequency equal to a resonance frequency of the analyte.

In some non-limiting examples, the first electromagnetic radiation is within the microwave range.

In some non-limiting examples, the frequency of the first electromagnetic radiation is swept over a target frequency range in which a resonant frequency of the analyte exists.

In some non-limiting examples, the first electromagnetic radiation has a frequency of more than 1 GHz, more than 3 GHz, more than 5 GHz, more than 8 GHz, less than 100 GHz, less than 50 GHz, less than 15 GHz, between 1 GHz and 100 GHz, between 3 GHz and 50 GHz, or between 5 GHz and 15 GHz.

In some non-limiting examples, the frequency of the third electromagnetic radiation is shifted from the second electromagnetic radiation by an amount equal to the resonance frequency of the analyte.

In some non-limiting examples, the method can include filtering out the third electromagnetic radiation from background electromagnetic radiation using a filter.

In some non-limiting examples, the filter can be an optical filter. Filtering out the third electromagnetic radiation from background electromagnetic radiation using the filter can occur before receiving the third electromagnetic radiation.

In some non-limiting examples, the third electromagnetic radiation is passed through a vapor cell that is a reference cell with an absorption line matched to the frequency of the second electromagnetic radiation.

In some non-limiting examples, the second electromagnetic radiation is within the visible light range or the infrared range.

In some non-limiting examples, the method can include determining a concentration of the analyte within the sample based on the third electromagnetic radiation.

In some non-limiting examples, at least a part of the providing the first electromagnetic radiation and providing the second electromagnetic radiation occur simultaneously.

In some non-limiting examples, determining the presence of the analyte includes comparing the spectrum of the received a third electromagnetic radiation to previously acquired spectra of a reference sample containing the analyte.

In some non-limiting examples, the analyte is a pathogen.

Some non-limiting examples of the disclosure provide a system for detecting an analyte within a sample. The system can include a first energy source configured to provide a first electromagnetic radiation to the sample in a manner that resonantly excites mechanical vibrations in the analyte, and a second energy source configured to provide a second electromagnetic radiation to the sample so as to interact with vibrating analyte. A third electromagnetic radiation can be produced based on the interaction. The system can include a receiver. The receiver can include a filter configured to separate the third electromagnetic radiation from background electromagnetic radiation, and a detector configured to receive the filtered a third electromagnetic radiation and produce a detection signal. The system can include a processor configured to provide information regarding the presence of the analyte based on the detection signal.

In some non-limiting examples, the system is integrated within at least one of a heating, ventilation, and air conditioning (HVAC) system, an ultraviolet (UV) disinfectant system, a filtering system, a fluid supply system that is a water supply system, or a duct.

In some non-limiting examples, the first energy source is a microwave emitter. The first electromagnetic radiation can be within the microwave range. The second energy source can be a laser emitter and the second electromagnetic radiation can be within the visible light range or the infrared range.

In some non-limiting examples, the filter is a vapor cell that is a reference cell with an absorption line matched to the frequency of the second electromagnetic radiation. The filter can be positioned in front of the detector.

In some non-limiting examples, the system can include a second receiver. The second receiver can include a second filter configured to separate the third electromagnetic radiation from background electromagnetic radiation, and a second detector configured to receive the filtered a third electromagnetic radiation and produce a detection signal. The second receiver can be positioned on the opposite side of the sample as the second energy source so as to receive transmitted second electromagnetic radiation.

Some non-limiting examples of the disclosure provide a computer-implemented method for determining a resonant frequency of an analyte. The method can include causing, using one or more computing devices, a first energy source to emit first electromagnetic radiation towards a sample that includes the analyte, causing, using the one or more computing devices, a second energy source to emit second electromagnetic radiation, different from the first electromagnetic radiation, towards the sample that includes the analyte, receiving, using a detector and the one or more computing devices, third electromagnetic radiation different from the first electromagnetic radiation and the second electromagnetic radiation, and determining, using the one or more computing devices, a resonant frequency of the analyte based on the received third electromagnetic radiation.

These and other advantages and features of the present invention will become more apparent from the following detailed description of the preferred aspects of the present invention when viewed in conjunction with the accompanying drawings.

### BRIEF DESCRIPTION OF THE DRAWINGS

The drawings described herein are for illustration purposes only and are not intended to limit the scope of the disclosed non-limiting examples in any way. The drawings described herein are for illustrative purposes only of selected non-limiting examples and not all possible implementations, and are not intended to limit the scope of any of the various non-limiting examples. It is understood that the drawings are not drawn to scale.

FIG. 1 is a flowchart setting forth some non-limiting example steps of a method of detecting an analyte within a sample, in accordance with one aspect of the present disclosure.

FIG. 2 is a schematic diagram of one example of a system for detecting an analyte within a sample, in accordance with one aspect of the present disclosure. For clarity purposes, electromagnetic radiation is not depicted.

FIG. 3 is a flowchart setting forth some non-limiting example steps of a method of detecting a multiple target pathogens in a subject, in accordance with one aspect of the present disclosure.

FIG. 4 is a flowchart setting forth some non-limiting example steps of a method of detecting SARS-CoV-2 within a subject, in accordance with one aspect of the present disclosure.

FIG. 5 is a flowchart setting forth some non-limiting example steps of a method of detecting a pathogen within a sample, in accordance with one aspect of the present disclosure.

FIG. 6 is a portion of a flowchart of a process for determining a resonant frequency of an analyte of a sample, and a process for building a database of spectra for analytes.

FIG. 7 is a portion of a flowchart of the process of FIG. 7.

FIG. 8 is a portion of a flowchart of a process for detecting an analyte within a sample.

FIG. 9 is a portion of a flowchart of the process of FIG. 8

FIG. 10 is a schematic diagram of the system of the experiment in Example 1.

FIG. 11 is a schematic diagram of the method steps and components of the system of the experiment in Example 1.

FIG. 12 is a depiction of a spherical coronavirus particle, a mass-spring system model, and a graphical representation of the modeled results of the experiment of Example 1.

FIG. 13 is a depiction of the interaction diagram of the laser probe beam with the sample in the experiment of Example 1. The spectrum of the laser probe beam are shown graphically at various stages within the detection process.

FIG. 14 is a depiction of the signal processing chain architecture of the experiment of Example 1.

FIG. 15 is a graph of the expected probability distribution for typical particles as a function of a virus diameter (“dᵥ”) relative to the effective speed of sound of the capsid protein surrounding the virus (“v_{c}”) parameter (e.g., the probability verses the ratio between the dᵥ and the v_{c}).

### DETAILED DESCRIPTION

Typical biological testing methods for a specific pathogen often involve determining a genetic sequence of components within the sample, detecting antigens (e.g., surface proteins) of the sample, or detecting antibodies against the specific pathogen in the sample. For the genetic sequence process, a sample from a subject is processed to extract the genetic material and convert it, if necessary (e.g., if the specific pathogen is a retrovirus), to deoxyribonucleic acid (“DNA”). Then, the DNA is analyzed using PCR, NEAR, etc., to determine the genetic sequence of the DNA and determine if this genetic sequence matches the genetic sequence of the specific pathogen. For the detecting antigens process, a portion of the sample is placed in contact with compounds that bind to the specific pathogen (e.g., labeled antibodies including fluorescent labeled antibodies). Then, if biding occurs between the antigen and the compound, a visible indication occurs (or this interacting can be detected by other instrumentation). Last, for the antibody process, a blood sample is taken from the subject, and is added to compounds that can include labeled antigens to the desired antibody to be tested. If antibodies are present within the blood, the antibodies bind to the compounds, which can be visually detected or detected by instrumentation.

While each of these processes can detect the specific pathogen, they have downsides. First, all of these processes require different reagents, and require a portion of the sample to be used up during the detection process. For example, some reagents may not be easily obtainable (e.g., expensive, distribution logistics issues including being in rural areas, etc.), and some can require substantial operator skill. As a more specific example, the processes needed to implement a PCR reaction can take substantial amounts of reagents (e.g., DNA polymerase), equipment (e.g., a centrifuge) that may not be available to particular areas, and operator skill needed to appropriately use the reagents and the equipment. In some cases, the scarcity of reagents including test strips for antigen or antibody tests can undesirably impact appropriate testing of populations. For example, in this case, testing may be restricted to individuals displaying symptoms to more efficiently utilize the scarce reagents. However, for pathogens including COVID-19 that have a relatively large population of asymptomatic carriers, testing would not be conducted on these individuals who may go on to infect individuals.

Second, some of these processes require substantial time resources, and some have a trade-off between time and accuracy. For example, for the PCR process, testing results may take a couple of days to receive, due to the time needed for reactions (e.g., the DNA generation) to occur. As another example, while the antigen and antibody tests can be much faster than the PCR process, the antigen and antibody tests are often unable to detect low levels of the virus, which could otherwise be detected by the PCR method. Thus, a negative test from the antigen or antibody test does not necessarily mean that the patient does not have the pathogen, but rather that the pathogen is at too low a level to be detected. As such, one of these patients could have the pathogen (e.g., when the test occurs early in the infection when pathogen levels area low), and rather than quarantining, could go on to transmit the pathogen to other individuals.

Third, each of these processes can undesirably use up a portion or the entire sample of the patient. For example, in the PCR process, the entire sample is used to conduct the sequencing, while for the antigen and antibody tests the entire sample (or a significant portion of the sample) is used up for the testing. Thus, typically the same sample cannot be run again, such as if an error in the processing has occurred (e.g., the operator used the incorrect reagent).

Fourth, each of these processes are only specific to a single pathogen. For example, supposing that a practitioner suspects that the patient's symptoms are caused by a specific pathogen, the practitioner can request that the patient be tested for the specific pathogen. However, supposing that the test resulted in a negative test for the patient (e.g., the patient does not have the specific pathogen), the practitioner (and the patient) still do not know the cause of the symptoms, and if the symptoms are caused by a different pathogen. Thus, this requires the practitioner to further evaluate and potentially request other tests to be conducted for different pathogens. As such, the patient may not be treated at the ideal time for other pathogens because the practitioner has not concluded if or what pathogen is causing the patient's symptoms.

Some non-limiting examples of the disclosure provide advantages to these issues (and others) by providing improved systems and methods for analyte detection. For example, as will be described, the present disclosure provides systems and methods for determining the presence of a vibrating analyte or pathogen by measuring a scattering process different from simple Brillouin scattering. In particular, the present disclosure provides systems and methods for modulating a medium or sample. Upon excitation of the medium or sample, a frequency shift is induced in the scattered light by the particular resonant vibration of the analyte or pathogen, if present. This frequency shift induced by the resonant vibration is a Doppler shift and manifests differently, in physics, from the phonon scattering of Brillouin processes.

This detection method herein provides advantages over the previously described reagent based detection methods. First, this detection method does not require the use of any reagent that binds to the particular pathogen in order to detect the pathogen (e.g., the binding of the reagent with the particular pathogen allowing for detection of the pathogen). Thus, no portion of the sample needs to be used up, the sample can be entirely sealed from the operator, and the sample can be retested as needed. This removes the limiting factor of availability of reagents, and thus more samples (e.g., from other individuals) can be tested. Second, this detection method can be significantly faster (e.g., orders of magnitude faster) than the other testing methods. For example, this testing method can be completed in less than 10 seconds, while the PCR process at best can be completed in hours (e.g., but typically requires at least a day), and the antigen (or antibody) tests at best can be completed in around 15 minutes. Thus, the testing method herein can provide significant improvements to testing throughput. Third, this testing method can be used for a number of pathogens, and not just a single pathogen as in the traditional testing methods. For example, because each specific pathogen can have a unique resonant frequency (e.g., a frequency “fingerprint”), this detection method can be used to detect other pathogens (e.g., by comparing the received data to example data from each known pathogen). Thus, this detection method herein can advantageously detect more than one pathogen.

In one non-limiting example, a system and method is provided for rapid (<10 sec) SARS-CoV-2 detection. The systems and methods provide markedly improved sensitivity over current methods (<5 viral copies/mL), excellent specificity, with minimal non-specific signal, and the ability to process any sample, including saliva, sputum, blood, breath exhalant, naso- and oropharyngeal swabs, stool, and surface swabs, in a contained manner, without special or additional specimen processing.

In one non-limiting example, systems and methods are provided to induce resonant ultrasonic mechanical vibration in SAR-CoV2 particles by irradiation with microwave energy at a frequency (˜8-15 GHz) tuned to the size, sound speed, and/or charge distribution of SAR-CoV2 virus. The systems and methods then facilitate the observation of the resultant viral sub-nanometer resonant vibration using electromagnetic radiation to determine the presence or absence of the virus. In one non-limiting example, a laser source can be used to interrogate the vibrating sample and the presence of absence of a Doppler shift induced by the resonant vibration of the virus indicates its presence or absence. In one example, the laser source can be carefully selected and a filter system matched to the laser source used to filter the wavelength of the laser source from detection, as well as other wavelengths, apart from the wavelength associated with the Doppler shift induced by the resonant vibration of the virus.

Though the material or analyte vibration produces a weak optical sideband, which would ordinarily be considered undetectable in the presence of the large amount of unwanted light scatter, the use of a laser frequency matched to the narrow atomic absorption line of a particular vapor cell, which has been demonstrated to filter unmodulated and unwanted background light remarkably effectively, yields sensitivity sufficient to identify the weak optical sideband(s) associated with the Doppler shift induced by the vibration of the target analyte, in this case, a virus. This enables extraordinarily sensitive photodetection of the weak signal from the vibrating virus. The excitation frequency can be selectively varied to selectively detect and classify viral particles by matching detected signals to known viral vibration spectra. In this manner, the proposed systems and methods provided herein can not only detect and classify SAR-CoV2 with sensitivity unmatched by existing alternative technologies but, can also be employed as a rapid diagnostic method for any of a variety of pathogens or, more generally, a select analyte.

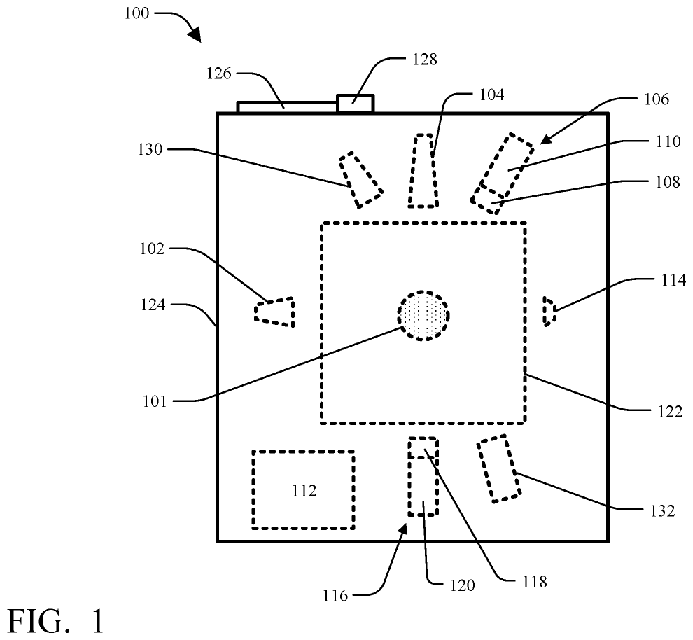

FIG. 1 is a schematic diagram of a system 100 for detecting an analyte within a sample 101 in accordance with the present disclosure. The system 100 can include a first energy source 102 configured to provide first electromagnetic radiation to the sample 101 in a manner that resonantly excites mechanical vibrations in the analyte and a second energy source 104 configured to provide second electromagnetic radiation to the sample 101 so as to interact with vibrating analyte, wherein third electromagnetic radiation is produced based on the interaction. The system 100 can also include a receiver 106 which includes a filter 108 configured to separate the third electromagnetic radiation from background electromagnetic radiation, and a detector 110 configured to receive the filtered third electromagnetic radiation and produce a detection signal. Additionally, a computing device 112 can be included and configured to provide information regarding the presence of the analyte based on the detection signal.

In some non-limiting examples, the first energy source 102 can be a radio wave transmitter that can emit radio waves (e.g., in a range from 30 Hertz (Hz) to 300 GHz). Thus, the first electromagnetic radiation can be within the range of radio waves. In some cases, the first energy source 102 can include oscillators, amplifiers, a power source (e.g., controllable by the computing device 112), an antenna, etc., and other components to cause the first energy source 102 to emit radio waves towards the sample 101. As a more specific example, the first energy source 102 can be a microwave transmitter, and thus correspondingly, the first electromagnetic radiation can be within the microwave range. In some non-limiting examples, the first electromagnetic radiation can have a substantially (i.e., deviating be less than 20 percent) uniform frequency. However, whether uniform or not, the first electromagnetic radiation can include a frequency equal to a resonance frequency of a specific, selected analyte (e.g., a virus, a bacteria, a fungi, etc.). For instance, the first electromagnetic radiation can have a frequency range within 2 GHz, 1 GHz, 0.5 GHz, 0.1 GHz, or 0.01 GHz of a resonance frequency of the analyte (e.g., a predetermined resonance frequency of the analyte).

In some configurations, the first electromagnetic radiation can be within the microwave range, where the microwave range is considered to be between 300 MHz and 300 GHz. For example, the first electromagnetic radiation can have a frequency of more than 1 GHz, more than 3 GHz, more than 5 GHz, more than 8 GHz, less than 100 GHz, less than 50 GHz, less than 15 GHz, between 1 GHz and 100 GHz, between 3 GHz and 50 GHz, or between 5 GHz and 15 GHz. In some non-limiting examples, the first energy source 102 can be configured to sweep the frequency of the first electromagnetic radiation over a target frequency range which includes a resonant frequency of the analyte. For example, the first energy source 102 can be configured to sweep the frequency of the first electromagnetic radiation at a rate of rate of at least 10 GHz/s, at least 5 GHz/s, at least 1 GHz/s, or at least 0.1 GHz/s over the target frequency range.

As shown in FIG. 1, there system 100 can include a first electromagnetic radiation receiver 114 configured to receive the first electromagnetic radiation from the first energy source 102 and determine information regarding a property of the first electromagnetic radiation. For example, the first electromagnetic radiation receiver 114 can be a radio wave receiver that is configured to receive the first electromagnetic radiation emitted by the first energy source 102. In this case, the radio wave receiver can include components to appropriately receive and amplify the signal, which can include an antenna, an amplifier, etc. As a more specific example, including when the first energy source 102 is a microwave transmitter, the first electromagnetic radiation receiver 114 can be a microwave receiver. In some non-limiting examples, the first electromagnetic radiation receiver 114 can be in electrical communication with the computing device 112, the first energy source 102, or both, and can be configured to provide feedback information regarding a property of the first electromagnetic radiation in order to control the first energy source 102. While FIG. 1 shows that the first electromagnetic radiation receiver 114 is aligned with the first energy source 102 and is positioned on the opposite side of the sample 101 from the first energy source 102, the first electromagnetic radiation receiver 114 can be positioned at different locations relative to the first energy source 102. For example, the first radiation receiver 114 can be positioned substantially orthogonal to the first energy source 102, angled relative to the first energy source 102, substantially parallel to the first energy source 102 (e.g., and out of alignment with the first energy source 102), etc.

In some non-limiting examples, the second energy source 104 can be a coherent light source configured to emit coherent light towards the sample 101. Thus, the second electromagnetic radiation, emitted by the second energy source 104, can be coherent light. For example, the second energy source 104 can be a laser emitter (or in other words a laser) that can be configured to emit coherent light towards the sample 101. In some cases, the second electromagnetic radiation can be within the visible light range or the infrared range, where the visible light range is considered to be between 380 and 700 nanometers and the infrared range is considered to be between 700 nanometers and 1 millimeter. In this case, for example, the second energy source 104 can be a cesium laser, a potassium laser, a sodium laser, a rubidium laser, an iodine laser, an argon laser, a helium laser, a carbon dioxide laser, etc. In some configurations, the second energy source 104 can be configured to provide the second electromagnetic radiation with a substantially uniform frequency that can be equal to an absorption line of a reference cell used to receive the third electromagnetic radiation. The second energy source 104 can be configured to provide the second electromagnetic radiation with a wavelength of substantially or identically 780 nanometers. In some cases, such as illustrated in FIG. 1, the first energy source 102 and the second energy source 104 can be arranged to provide the first electromagnetic radiation and the second electromagnetic radiation sample in an orthogonal direction to the sample. For example, the first energy source 102 can be orthogonal to the second energy source 104.

In some non-limiting examples, the receiver 106 can be configured to receive the third electromagnetic radiation. In some cases, the receiver 106 can be positioned on the same side of the sample 101 as the second energy source 104 so as to receive the third electromagnetic radiation that has been scattered. In some cases, the receiver 106 can be angled relative to the second energy source 104. For example, the receiver 106 can be obliquely angled at an acute angle relative to the second energy source 104. The detection signal produced by the receiver 106 can include information on the spectrum of the third electromagnetic radiation. For example, the receiver 106 can receive the third electromagnetic radiation, which can be used (e.g., by the computing device 112) to generate a spectrum (e.g., a frequency or wavelength spectrum). In this way, the spectrum can be compared (e.g., by the computing device 112) to a reference spectrum to determine whether or not an analyte (at a specific concentration) is present in the sample 101. As shown in FIG. 1, the receiver 106 can include a filter 108 and a detector 110. The filter 108, which can be an optical filter, can be aligned with the detector 110 and can be positioned at an end of the detector 110 that faces the sample 101. The filter 108 can be configured to block transmission of electromagnetic radiation (e.g., within a range that includes the second electromagnetic radiation) from being received by the detector 110, while allowing electromagnetic radiation outside of the range to be received by the detector 110. Thus, for example, the filter 108 can include a reference cell with an absorption line matched to the frequency of the second electromagnetic radiation. In some cases, the reference cell can be a vapor cell including a potassium vapor cell, a sodium vapor cell, a rubidium vapor cell, or an iodine vapor cell. As a more specific example, the filter 108 can include a reference cell that is a rubidium 780 nm vapor cell and the second electromagnetic radiation can have a substantially uniform wavelength of 780 nm. Regardless of the configuration, the filter 108 can function as a band stop filter (e.g., a notch filter) with a center frequency that can include the frequency of the second electromagnetic radiation.

In some non-limiting examples, the detector 110 can be a photodetector that can be configured to sense electromagnetic radiation within a range of wavelengths (and frequencies). For example, the detector 110 can sense electromagnetic radiation within the visible light range, within a wavelength range of 380 nm to 890 nm. In some cases, the detector 110 can have photon counting capabilities (e.g., the detector 110 is able to sense each photon and determine its corresponding energy). In some specific cases, the detector 110 can be a photodetector with a photon counting head having photomultiplier tubes (e.g., a Hamamatsu H7421-50 photon counting head).

In some non-limiting examples, the system 100 can include a second receiver 116, which can be similar to the first receiver 106, but can be positioned at a different location relative to the second energy source 104 than the first receiver 116. For example, the second receiver 116 can be aligned with the second energy source 104 and can be positioned on an opposite side of the sample 101 as the second energy source 104. In some cases, however, the second receiver 116 can be substantially parallel to the second energy source 104, orthogonal to the second energy source 104, or angled relative to the second energy source 104. In some specific cases, the second receiver 116 can be aligned with the first receiver 106, in which the second receiver 116 can be parallel to the first receiver 106. In some non-limiting examples, the distance between the receiver 106 and the sample 101 (or holder that receives the sample 101) can be substantially or identically equal to the distance between the receiver 116 and the sample 101 (or the holder that receives the sample 101). In this way, because the optical data received by each receiver 106, 116 should be substantially similar, the optical data can be combined (e.g., added together, averaged, etc.) between the receivers 106, 116 as appropriate to increase the detection sensitivity of the system 100. In addition, while two detectors 110, 120 are illustrated in FIG. 1, the system 100 can have additional detectors (e.g., and additional one, two three, etc., detectors) each implemented in a similar way as the previously described detectors. In some cases, a first additional detector can be orthogonal to the detector 120, while a second additional detector can be orthogonal to the detector 120 (and parallel to the first detector).

In some non-limiting examples, the second receiver 116 can be positioned on the opposite side of the sample 101 as the second energy source 104 so as to receive transmitted second electromagnetic radiation, as shown in FIG. 1. That is, the first detector 110 is illustrated as being positioned to receive forward scatter, while the second detector 120 is positioned to receive backscatter. Both the first detector 110 and the second detector 120 can be utilized to capture both forward scatter and backscatter. The signals from the first detector 110 and the second detector 120 can be separately processed by the computing device 112 or can be summed prior to processing, such as to increase signal-to-noise ratio (“SNR”). Also, only one of the first detector 110 or the second detector 120 can be included or utilized to capture only forward scatter or only backscatter, respectively.

In some non-limiting examples, the receiver 116 can also include a second filter 118, and a second detector 120, with the second filter 118 aligned with and positioned at an end of the detector that faces the sample 101. The second filter 118 can be configured to separate the third electromagnetic radiation from background electromagnetic radiation, and the second detector 120 can be configured to receive the filtered third electromagnetic radiation and produce a transmitted detection signal. For example, the filter 118 can block passage of electromagnetic radiation within a frequency range that includes the frequency of the second electromagnetic radiation. The detector 120 can be implemented in a similar manner as the detector 110. For example, the detector 120 can be a photodetector with photon counting capabilities.

In some configurations, a lock-in technique can be utilized with the above-described system 100, where a control source 130 is included to deliver a control signal that is phased to the oscillations of the analyte (e.g., the microwave-induced oscillations of the analyte). The control signal, thereby, forms a reference signal and a lock-in detector 132 is provided to look for signal oscillations in phase with the reference signal. In this way, further signal information is acquired that can be used to increase SNR. Alternatively, this lock-in technique and reference signal could be the second electromagnetic radiation.

The system 100 can further include a sample holder 122 configured to maintain a constant position of a portion of the sample 101, where the portion of the sample 101 is within the optical pathways of the first electromagnetic radiation and the second electromagnetic radiation. For example, the sample holder 122 can be a container that (fully or partially) encloses and seals the sample 101 within the interior volume of the sample holder 122. In some cases, the sample holder 122 can be a cuvette (or other container) with a plug that seals the sample 101 contained within the cuvette (or other container) from the ambient environment. The sample holder 122 can include walls that allow the first, second, and third electromagnetic radiation to pass through, and the walls of the sample holder 122 can be transparent to allow these electromagnetic radiations to pass through. In some cases, the sample holder 122 can be contained within and supported by a housing 124 of the system 100. For example, the sample holder 122 can be removably coupled to the housing 124. As a more specific example, the sample holder 122 can be received in a well of the housing 124 to support the sample holder 122 (and thus the sample 101) during analysis and detection.

In some non-limiting examples, when the sample holder 122 fully encloses the sample 101, there is little to no risk for transmission of a potential pathogen of the sample 101 to an operator handling the sample holder 122. In addition, because the sample 101 is not used up during analysis (e.g., a portion of the sample 101 is not used to react with a detecting agent, such as a detection antibody that binds to a pathogen), the operator can be shielded from the transmission of potential pathogens in the sample 101, and the sample 101 can be tested again, if desired.

In some non-limiting examples, the sample holder 122 can be formed of a material designed to control or reduce the Brillouin signal. For example, the holder 122 can be formed of a material that has a Brillouin frequency range that is separated from or distinct from the third electromagnetic radiation. As a more specific example, Aerogel can be used which has a much lower sound speed than typical bio-materials and, thus, a different Brillouin frequency range. Additionally or alternatively, the sample 101 can be a gaseous sample that is collected and held or passed through the system 100. In this manner, the holder 122 can be a chamber or structure that is configured to receive a sample that includes (or does not include) the analyte. For example, the holder 122 can be fully enclosed, or can be partially enclosed (e.g., with opposing ends open, or having holes therethrough). In this way, the sample 101 can be a fluid (e.g., a gas including aerosols, other particulates, etc.) and thus the system 100 can be configured to sample and test air in a given space or room, or continuously monitor for a specific analyte. Furthermore, in this regard, the system 100 can serve to control or provide feedback to other systems, such as HVAC systems, UV cleaners, filtering systems, water delivery systems, vents, ducts, or other systems designed to control or process the detected analyte or pathogen. In this case, for example, the system 100 can be integrated within these systems, and because the sample can be a volume of fluid (e.g., air), the housing 124 can be omitted, or the housing 124 can be in fluid communication with a fluid of one of the these systems (e.g., an HVAC system).

As shown in FIG. 1, the energy sources 102, 104, and the receivers 106, 116 can be positioned within the interior volume of the housing 124. In addition, the energy sources 102, 104 and the receivers 106, 116 can be positioned so that each of these can surround the sample 101 (and the sample holder 122). In other words, the energy sources 102, 104 and the receivers 106, 116 can be positioned radially away from the sample 101(and the sample holder 122). In some non-limiting examples, the interior volume of the housing 124 can be sealed from the ambient environment, which can prevent ambient light from being emitted into the interior volume. In this way, the housing 124 can be formed out of materials that do not allow passage of light therethrough (e.g., metals, reflective surfaces, etc.). In some non-limiting examples, the interior of the housing 124 can include an electromagnetic shield (e.g., a sheet metal cage) that surrounds and encloses the energy sources 102, 104, and the receivers 106, 116. In this way, the electromagnetic radiation produced by the energy sources 102, 104 does not escape outside of the housing 124 (and into the ambient environment). While the housing 124 is illustrated as being a prism (e.g., a cube, a rectangular prism, etc.), the housing 124 can have other shapes (e.g., a cylinder, a sphere, etc.).

In some non-limiting examples, the interior volume of the housing 124 can be sealed during analysis of the sample 101 (e.g., delivery of one or more electromagnetic radiations). In some cases, the housing 124 can include a cover that is pivotally coupled to a base that hingedly swings open to allow access to the base that can support the same holder 122 and the sample 101. Then, once the sample holder 122 and the sample 101 is placed on the base, the cover can rotate to enclose the sample 101 from the ambient environment. In other configurations, the system 100 can include a door that is pivotally coupled to the housing 124, which can rotate to allow access to the interior volume of the housing 124, and to block access to the interior volume of the housing 124 (e.g., by enclosing and isolating the interior volume, including the sample 101, from the ambient environment).

In some non-limiting examples, the computing device 112 can be in communication with some or all of the components of the system 100. For example, the computing device 112 can send instructions to and receive data from particular components of the system 100. As a more specific example, the computing device 112 can cause components of the system 100 to implement a particular task, including causing the energy sources 102, 104, and to emit or stop emitting electromagnetic energy, causing the energy sources 102, 104 to adjust the intensity of the respective electromagnetic energies. As another specific example, the computing device 112 can receive optical data from each of the detectors 110, 120. The computing device 112 can be configured to implement some or all of the processes described below, as appropriate. For example, the computing device 112 can be configured to determine a presence and a concentration of an analyte within the sample 101, based on the third electromagnetic radiation. In some cases, this can include the computing device 112 comparing the spectrum of the received third electromagnetic radiation to previously acquired spectra of a reference sample containing the analyte in order to determine the information regarding the presence or concentration of the analyte within the sample 101.

The computing device 112 can be implemented in different ways. For example, the computing device 112 can include typical components used such as a processor, memory, a display, inputs (e.g., a keyboard, a mouse, a graphical user interface, a touch-screen display, etc.), communication devices, etc. In some cases, the computing device 112 can simply be implemented as a processor. The computing device 112 can communicate with other computing devices and systems, including a server, a database, an electronic health records network, etc.

In some non-limiting examples, the system 100 can include a display 126 in electrical communication with the computing device 112 and configured to display information regarding the presence of the analyte within the sample 101. The system 100 can include an interface 128 in electrical communication with the first energy source 102, the second energy source 104, the receiver 106, and the computing device 112. The interface 128, which can be a user input (e.g., a touch screen, an actuatable button, etc.), can be configured to allow a user to control operating parameters of the first energy source 102 or the second energy source 104, and to adjust processing parameters of the computing device 112, or both. In some configurations, the interface 128 can be integrated into the display 126.

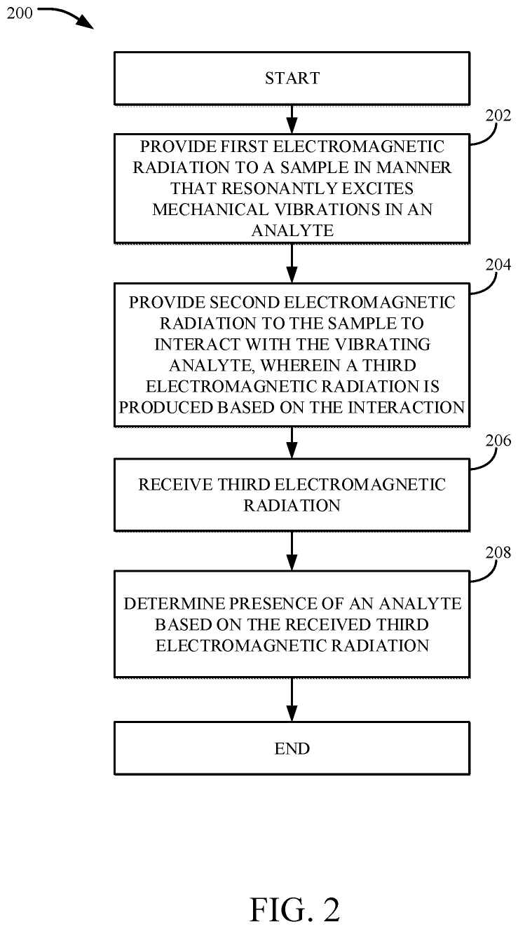

FIG. 2 is a flow chart setting forth some steps of a method 200 of detecting an analyte within a sample in accordance with the present disclosure. The method 200 includes a first step 202 of providing first electromagnetic radiation to the sample in a manner that resonantly excites mechanical vibrations in the analyte. The method continues with a second step 204 of providing second electromagnetic radiation to the sample so as to interact with a vibrating analyte, whereby third electromagnetic radiation is produced based on the interaction (e.g., between the second electromagnetic radiation and the vibrating analyte). Then, the method includes a third step 206 of receiving the third electromagnetic radiation, and a fourth step 208 of determining the presence of the analyte based on the received third electromagnetic radiation.

The first electromagnetic radiation provided in the method step 202 can have a substantially uniform frequency equal to a resonance frequency of the analyte. For instance, the first electromagnetic radiation can have a frequency range within 2 GHz, 1 GHz, 0.5 GHz, 0.1 GHz, or 0.01 GHz of a resonance frequency of the analyte. The first electromagnetic radiation can be within the microwave range, where the microwave range is considered to be between 300 MHz and 300 GHz. The first electromagnetic radiation can specifically have a frequency of more than 1 GHz, more than 3 GHz, more than 5 GHz, more than 8 GHz, less than 100 GHz, less than 50 GHz, less than 15 GHz, between 1 GHz and 100 GHz, between 3 GHz and 50 GHz, or between 5 GHz and 15 GHz. In order to ensure mechanical vibrations are excited in the analyte, the frequency of the first electromagnetic radiation can be swept over a target frequency range in which a resonant frequency of the analyte exists. The frequency of the first electromagnetic radiation is swept at a rate of at least 10 GHz/s, at least 5 GHz/s, at least 1 GHz/s, or at least 0.1 GHz/s over the target frequency range. The target frequency range can be predetermined using prior experimental results.

The second electromagnetic radiation provided in the method step 202 can be within the visible light range or the infrared range, where the visible light range is considered to be between 380 and 700 nanometers and the infrared range is considered to be between 700 nanometers and 1 millimeter. The second electromagnetic radiation can have a substantially uniform frequency equal to an absorption line of a reference cell used to receive the third electromagnetic radiation in method step 204. The second electromagnetic radiation can specifically have a wavelength of 780 nanometers. The first electromagnetic radiation can be provided to the sample in an orthogonal direction to the second electromagnetic radiation.

The third electromagnetic radiation of method step 204 can be produced by the second electromagnetic radiation scattering off of the vibrating analyte and undergoing a phase modulation and a spectrum change. Specifically, the frequency of the third electromagnetic radiation can be Doppler shifted from the second electromagnetic radiation by an amount equal to the resonance frequency of the analyte. Explained another way, the first electromagnetic radiation and the second electromagnetic radiation can be selected or designed to achieve an amplitude or phase modulation of the second electromagnetic radiation. In particular, the first electromagnetic radiation and the second electromagnetic radiation can be selected or designed such that their interaction at the analyte produces Stokes and anti-Stokes sidebands to the second electromagnetic radiation that are in phase or coherent with each other. In both traditional Raman, Brillouin, CARS and four-wave mixing processes, the stokes and anti-stokes are not in phase because they are produced by light interacting with a thermally stimulated field. However, in the systems and methods provided herein, the second electromagnetic radiation can interact with a non-thermally stimulated first electromagnetic radiation to produce an acoustic field where the forward and backward traveling waves are coherent and, therefore, produces an optical field whose sidebands are also coherent, which is one way to form the third electromagnetic radiation. Thus, both + and − Doppler shifts are present due to the coherence between the Stokes and anti-Stoke wave.

The method step 206 of receiving the third electromagnetic radiation can include filtering to reduce radiation that is not the third electromagnetic radiation and/or measuring the spectrum of the third electromagnetic radiation. The method step of receiving the third electromagnetic radiation can comprise filtering out background electromagnetic radiation or radiation that is not the third electromagnetic radiation. The background electromagnetic radiation can generally include the second electromagnetic radiation, including a portion of the second electromagnetic radiation that has scattered off of components of the sample other than the analyte. In order to filter out this background electromagnetic radiation, the third electromagnetic radiation can be passed through a reference cell with an absorption line matched to the frequency of the second electromagnetic radiation. The reference cell can comprise or can be selected from the group consisting of a cesium vapor cell, a potassium vapor cell, a sodium vapor cell, a rubidium vapor cell, and an iodine vapor cell. In one non-limiting example, the reference cell can be a rubidium 780 nm vapor cell and the second electromagnetic radiation can have a substantially uniform wavelength of 780 nm.

The method 200 can optionally comprise an additional method step of determining a concentration of the analyte within the sample based on the third electromagnetic radiation. That is, the method 200 can simply detect a presence or absence of the analyte, or can alternatively or additionally include determining a concentration of the analyte. The method 200 can further comprise an additional method step of determining a quantity of the analyte within the sample based on the third electromagnetic radiation. For instance, if the analyte was a pathogen, this additional method step can determine a number of individual viral particles, or virions, present within the sample, or a concentration of individual viral particles in the sample.

In the method 200, at least a part of the method step 202 and method step 204 can occur simultaneously. As an example, the second electromagnetic radiation can be provided to the sample in isolation for a period of time before the first electromagnetic radiation is then provided simultaneously with the second electromagnetic radiation. Providing only one of the first electromagnetic radiation or the second electromagnetic radiation can allow for a baseline background electromagnetic radiation reading to be received. The method 200 can allow for rapid detection of the analyte. For instance, method steps 202, 204, and 206 can all occur within the same time period, where the time period lasts less than 120 seconds, less than 60 seconds, less than 30 seconds, less than 15 seconds, or less than 10 seconds.

The method 200 can include determining the presence of the analyte by comparing the spectrum of the received third electromagnetic radiation to previously acquired spectra of a reference sample containing the analyte. The analyte can be a pathogen. The analyte can be a pathogen that is SARS-CoV-2.

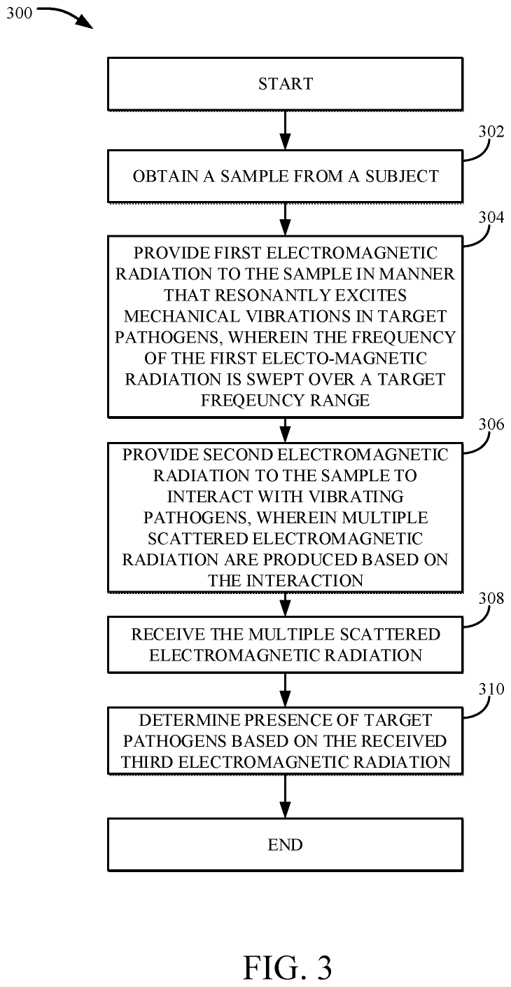

FIG. 3 is a flow chart setting forth some steps of a method 300 of detecting multiple target pathogens in a subject in accordance with the present disclosure. As one non-limiting example, the system of FIG. 2 can be used with the method 300. The method 300 includes a first step 302 of obtaining a sample from the subject. The method also includes a second step 304 of providing first electromagnetic radiation to the sample in a manner that resonantly excites mechanical vibrations in the target pathogens. The frequency of the first electromagnetic radiation is swept over a target frequency range in which resonant frequencies of the target pathogens exist. The method further includes a third step 306 of providing second electromagnetic radiation to the sample so as to interact with vibrating target pathogens, whereby multiple scattered electromagnetic radiation are produced based on the interaction. The method further includes a fourth step 308 of receiving the multiple scattered electromagnetic radiation, and a fifth step 310 of determining the presence of the target pathogens based on the received multiple scattered electromagnetic radiation. The method 300 can include any of the elements previously described herein for the method 200.

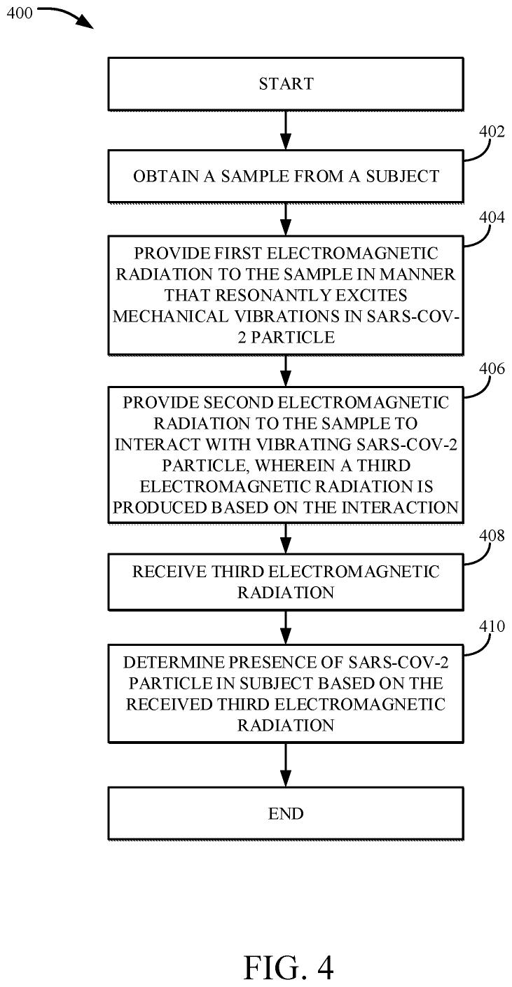

FIG. 4 is a flow chart setting forth some steps of a method 400 of detecting SARS-CoV-2 within a subject in accordance with the present disclosure. The method 400 includes a first step 402 of obtaining a sample from the subject and a second step 404 of providing first electromagnetic radiation to the sample in a manner that resonantly excites mechanical vibrations SARS-CoV-2 particle. The method 400 further includes a third step 406 of providing second electromagnetic radiation to the sample so as to interact with vibrating SARS-CoV-2 particle, wherein third electromagnetic radiation is produced based on the interaction. The method 400 also includes a fourth step 408 of receiving the third electromagnetic radiation; and a fifth step 410 of determining the presence of SARS-CoV-2 within the subject based on the received third electromagnetic radiation. The method 400 can include any of the elements previously described herein for the method 200.

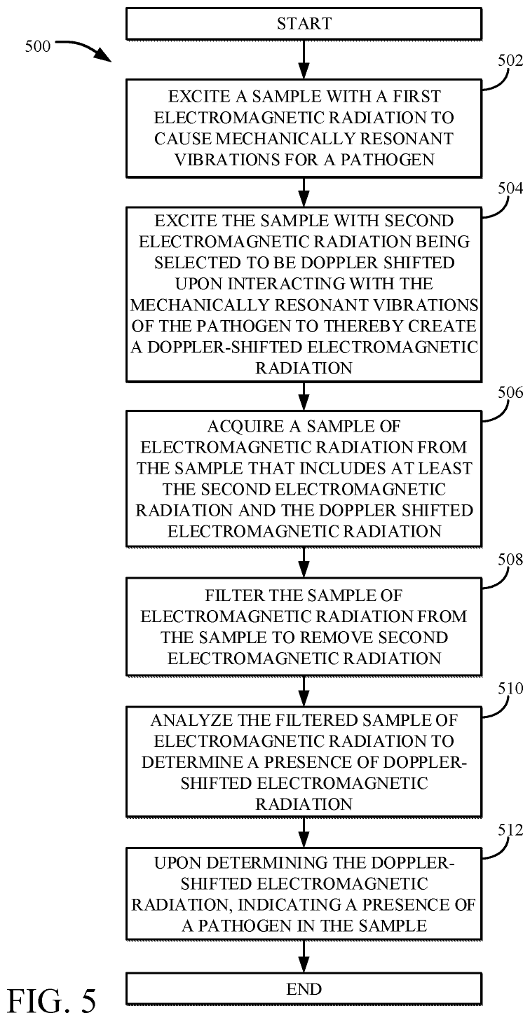

FIG. 5 is a flow chart setting forth some steps of a method 500 of detecting a pathogen within a sample in accordance with the present disclosure. The method 500 includes a first step 502 of exciting the sample with a first electromagnetic radiation selected to cause mechanically resonant vibrations in the pathogen. The method continues at step 504 with exciting the sample with a second electromagnetic radiation having a selected frequency configured to be Doppler shifted upon interacting with the mechanically resonant vibrations of pathogen to thereby create a Doppler-shifted electromagnetic radiation. At step 506, the method 500 includes acquiring a sample of electromagnetic radiation from the sample that includes at least the second electromagnetic radiation and the Doppler-shifted electromagnetic radiation. The method 500 includes filtering the sample of the electromagnetic radiation from the sample to remove the second electromagnetic radiation, at step 508 and analyzing the filtered sample of the electromagnetic radiation to determine a presence of the Doppler-shifted electromagnetic radiation at step 510. Finally, step 512 includes indicating the presence of the pathogen in the sample upon determining the Doppler-shifted electromagnetic radiation.

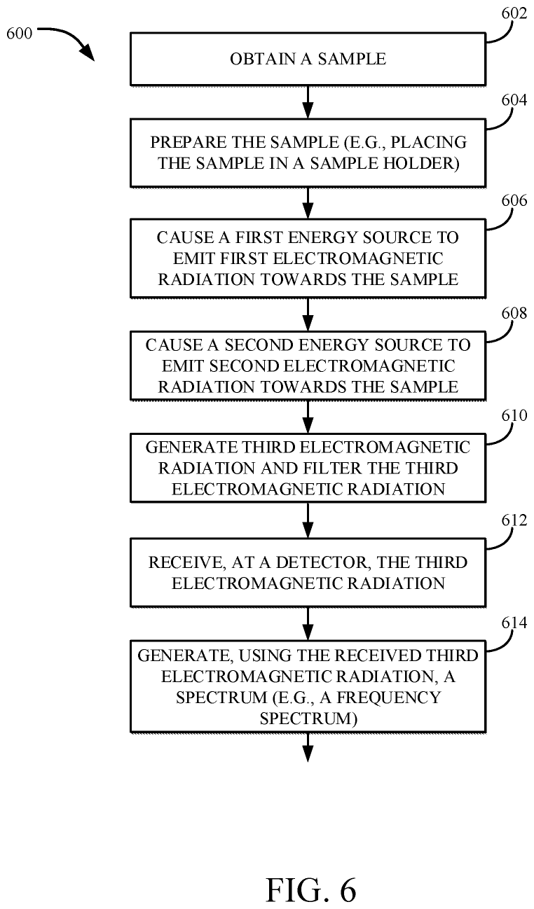

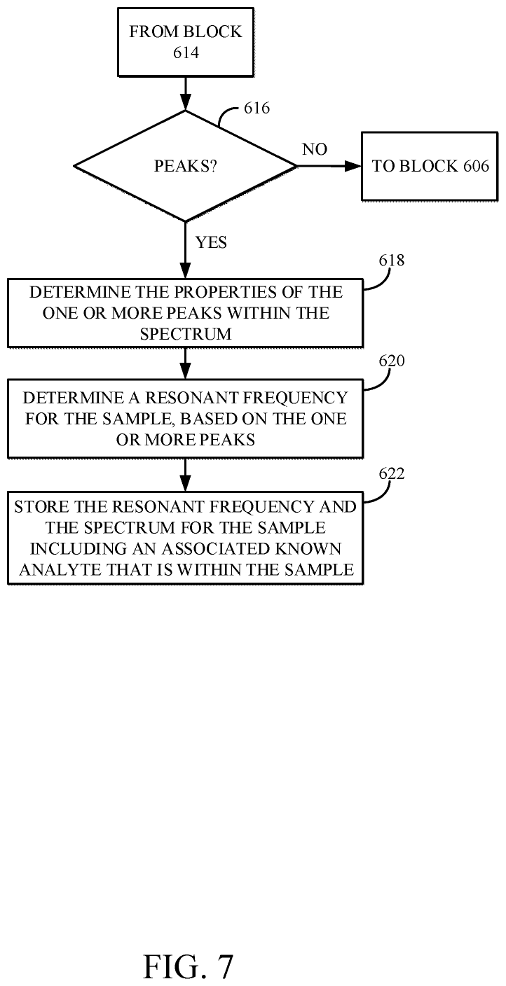

FIGS. 6 and 7 shows a flowchart of a process 600 for determining a resonant frequency of an analyte of a sample, and a process for building a database of spectra for analytes. Each of these processes can be implemented using the previously described systems (e.g., the system 100). In addition, some or all of the blocks of the process 600 can be implemented using one or more computing devices (e.g., the computing device 112), as appropriate. In some cases, the analyte can be a biological analyte including a virus, a bacteria, a fungi, a eukaryote, etc.

At 602, the process 600 can include obtaining a sample (e.g., from a subject), which can be similar to the block 302 of the process 300 (and the block 402 of the process 400). In some cases, this can include swabbing a portion of the subject with a swab (e.g., a cotton based or other absorbing swab). For example, the swab can be brought into contact with a portion of the subject (e.g., the nose cavity of the subject, the oral cavity of the subject, etc.) to wick away a bodily fluid (e.g., saliva, spit, etc.). In some cases, the swab having the bodily fluid absorbed thereon can be placed into a solution (e.g., water, water with solutes dissolved therein) with a known volume and composition (e.g., the amount of chemicals dissolved therein) to force at least some of the bodily fluid (and potential analytes, or pathogens) into the solution. In other cases, the block 602 can include receiving a bodily fluid sample (e.g., blood) with a known volume from the patient (e.g., collecting the bodily fluid sample from the subject). In some cases, the subject can be from a living subject including a human, livestock (e.g., a chicken, a cow, a pig, etc.), etc. In other cases, the sample can be from an inanimate object (e.g., dirt, fluid including an air flow, soil, etc.). For example, the inanimate object can be partially (or entirely) submerged into a solution to prepare the sample to be tested.

At 604, the process 600 can include preparing the sample. In some cases, this can include placing the sample into a sample holder. For example, this can include loading (e.g., with a pipette) the entire or a portion of the sample into the sample holder. In some cases, this can include loading the entire or a portion of the solution in which the sample has been submerged into the sample holder. In some configurations, this can include placing the sample (including the sample holder) into the housing of a system (e.g., the housing 124 of the system 100). For example, this can include isolating the sample from the ambient environment when the sample is placed within the housing (e.g., by closing a door of the housing, or placing a cover around the sample, etc.). In some cases, the block 604 can include a computing device receiving an indication (e.g., a user input from a user interface) that the sample is loaded within the housing. The block 604 can also include a computing device receiving a volume of the sample (e.g., which can be used for concentration analysis).

At 606, the process 600 can include a computing device causing a first energy source to emit first electromagnetic radiation towards the sample, which can be similar to the blocks 202, 304, 404, 502 of the previous processes. In some cases, the first electromagnetic radiation can include multiple different frequencies within a frequency range, which can be from 30 Hz to 300 GHz, from 300 MHz to 300 GHz, etc. For example, the first electromagnetic radiation can have multiple light emissions with different frequencies that can be separated by a substantially uniform separation frequency. For example, a computing device can cause the first energy source to emit a first light emission at a first frequency within the frequency range, to emit a second light emission with a second frequency within the frequency range, and to emit a third light emission with a third frequency within the frequency range, and so on. In this case, the first frequency can be separated from the second frequency by substantially the same frequency step as the second frequency is separated by the third frequency. In some cases, the first, second, and third frequency can be integer multiples of a common frequency. In some configurations, the first, second, and third light emissions can be separated by a time delay. For example, after the first light emission has been emitted, a time delay (e.g., less than or equal to 1 second, less than or equal to 0.1 seconds, etc.) can be implemented (e.g., waited by the computing device) before emitting the second light emission. Regardless of the configuration, a computing device can cause the first energy source to emit the first electromagnetic radiation that sweeps over different frequencies within a frequency range.

In some non-limiting examples, a computing device only begins implementing the block 604 after the computing device receives the indication that the sample is loaded within the housing (e.g.. and the housing has been sealed from the ambient environment). In this way, electromagnetic radiation is not inadvertently emitted into the ambient environment.

In some non-limiting examples, at the block 606 the process 600 can include mechanically vibrating an analyte (e.g., a pathogen) within the sample at a resonant frequency that is characteristic of the analyte. For example, if the first electromagnetic radiation includes a light emission that includes the resonant frequency of the analyte, the analyte will begin vibrating at its resonant frequency. In some cases, this resonant frequency can be a fundamental frequency and the analyte can vibrate at a first harmonic of the fundamental frequency, a second harmonic of the fundamental frequency, etc.

At 608, the process 600 can include a computing device causing a second energy source to emit a second electromagnetic radiation towards the sample, which can be similar to the blocks 204, 306, 406, 504 of the previously described processes. In some cases, the second electromagnetic radiation can have a substantially uniform frequency. For example, the second electromagnetic radiation can be coherent light (e.g., in which case the second energy source is a coherent light source).

At 610, the process 600 can include generating third electromagnetic radiation. In some cases, if at the block 606, the first electromagnetic radiation resonantly vibrated the analyte, the second electromagnetic radiation interacts with the resonantly vibrating analyte to generate the third electromagnetic radiation. However, if at the block 606, the first electromagnetic radiation does not resonantly vibrate the analyte, then the third electromagnetic radiation is not generated. In some cases, generating the third electromagnetic radiation includes Doppler shifting the second electromagnetic radiation based on the presence of the resonantly vibrating analyte. For example, this can include Doppler shifting (in both directions) the frequency of the second electromagnetic radiation by the resonant frequency of the vibrating analyte to generate third electromagnetic radiation. Thus, the third electromagnetic radiation can have a positive Doppler shift and a negative Doppler shift of the same frequency relative to the frequency of the second electromagnetic radiation. In some cases, the third electromagnetic radiation can have frequencies that are Doppler shifts, in both directions, of integer multiples of the fundamental frequency of the vibrating analyte. In other words, the third electromagnetic radiation can have Doppler shifts that correspond to the harmonic frequencies of the vibrating analyte.

At 610, the process 600 can include filtering (e.g., using a band stop filter with a center frequency that includes the frequency of the second electromagnetic radiation) the third electromagnetic radiation (e.g., before being received by a detector). In some cases, the third electromagnetic radiation can include the second electromagnetic radiation (e.g., scattered electromagnetic radiation). Thus, the third electromagnetic radiation, which can include the second electromagnetic radiation can be passed through a filter to allow passage of the third electromagnetic radiation, but blocking passage of the second electromagnetic radiation through. In some cases, the filter can be a band stop filter having a center frequency that includes the frequency of the second electromagnetic radiation. In some specific cases, the filter can be a vapor reference cell (e.g., a vapor wavelength reference cell) having a characteristic absorption light frequency that overlaps with frequency of the second electromagnetic radiation. In this way, as the second electromagnetic radiation passes through the filter, the filter absorbs the second electromagnetic radiation so that the second electromagnetic radiation is largely undetected by the detector.

At 612, the process 600 can include a computing device receiving, at a detector, the third electromagnetic radiation, which can be similar to the blocks 206, 308, 408, 506 of the previous processes. In some configurations, the block 612 can include a computing device receiving at another detector the third electromagnetic radiation (e.g., which can also be filtered). In some cases, at the block 612, the process 600 can include a computing device receiving, at a detector, background electromagnetic radiation when the second electromagnetic radiation is not being emitted (and the first electromagnetic radiation is not being emitted).

At 614, the process 600 can include a computing device, generating, using the received third electromagnetic radiation, a spectrum. In some cases, this can be a frequency spectrum (e.g., the intensity relative to the frequency), a wavelength spectrum (e.g., the intensity relative to the frequency), etc. In some non-limiting examples, this can include a computing device combining (e.g., adding, averaging, etc.) first optical data from the third electromagnetic radiation received at a first detector, and second optical data from the third electromagnetic radiation received at the another detector. In other cases, this can include a computing device generating a first spectrum from the first optical data, generating a second spectrum from the second optical data, and combining the first spectrum with the second spectrum. In some cases, combining the first spectrum with the second spectrum can include a computing device adding the first and second spectrums together, or averaging the first and second spectrums together. In some cases, the computing device can generate the spectrum, based on the received background electromagnetic radiation. For example, the computing device can generate a first spectrum from the received third electromagnetic radiation, and can generate a background spectrum from the received background electromagnetic radiation. Then, in some cases, the computing device can subtract the first spectrum from the background spectrum to yield a resulting spectrum.

At 616, the process 600 can include a computing device determining whether or not any peaks are present within the spectrum. For example, the computing device can determine that a peak is present within the spectrum by determining if the peak is greater than a threshold value. In some cases, a computing device can determined the threshold value based on the background electromagnetic radiation. For example, the computing device can determine the average intensity of the background spectrum, and can use this as the threshold value. In other cases, such as when the first spectrum is subtracted from the background spectrum, the threshold value can be zero. The computing device can utilize this process to determine if any peaks are present, and if they occur, the frequency (or wavelength) that each of the peaks are positioned. In some cases, if at the block 616, a computing device determines that no peaks are present within the spectrum, the process 600 can proceed back to the block 606. In this case, the computing device can emit another first electromagnetic radiation, different from the first electromagnetic radiation, in hopes of mechanically resonating the analyte within the sample. However, if at the block 616, a computing device determines that there is at least one peak within the spectrum, the process can proceed to block 618.

At 618, the process 600 can include a computing device determining one or more properties of the one or more peaks within the spectrum. This can include a computing device determining a frequency for each peak (e.g., the frequency that overlaps with the respective peak), determining a height of each peak, etc. In some cases, this can include determining a width of each peak, which can be defined between respective ends of the peak which are above the same threshold. In this way, the computing device can determine a shape (e.g., a broadness) of each peak. In some cases, the determined peaks can be harmonic peaks. For example, a harmonic peak can be a peak that has a frequency that includes the frequency of the second electromagnetic radiation and integer multiples of the frequency of the first electromagnetic radiation. In other words, a harmonic peak can be separated from a fundamental frequency peak by an integer multiple of the frequency of the first electromagnetic radiation.

At 620, the process 600 can include a computing device determining a resonant frequency for an analyte within the sample, based on the one or more peaks. For example, the computing device can determine the resonant frequency by subtracting the frequency of peak having the highest amplitude from the frequency of the second electromagnetic radiation. As another example, the computing device can average the frequency of the two peaks having the highest amplitude (e.g., intensity). As yet another example, the computing device can determine a reflection axis (e.g., a vertical reflection axis) that one pair (or multiple pairs) of peaks are reflected about so that for each pair this distance from the reflection axis and each peak is substantially the same.

At 622, the process 600 can include a computing device storing the resonant frequency and the spectrum for the sample in another computing device (e.g., a server, a database of a sever, etc.). In some cases, this can include a computing device receiving a known analyte (e.g., a pathogen) that is within the sample, associating the known analyte with the resonant frequency of the sample and the corresponding generated spectrum, and storing the associated known analyte with the corresponding data (e.g., the resonant frequency, the generated spectrum, etc.). In some cases, the computing device can store characteristics of the sample related to the concentration of the sample including the height of the one or more peaks (e.g., the peak with the highest amplitude), the volume of the sample, a known concentration of the analyte within the sample, properties of the sample including the amount of solute within the sample, the pH of the sample, etc. In this way, a computing device can determine (and store) a concentration curve for a specific analyte. In this case, a computing device can utilize the some or all of the blocks 602-622 of the process 600 for different samples having different known concentrations (e.g., dilutions, such as serial dilutions) of the same analyte with other characteristics of the sample remaining constant throughout the different samples (e.g., type of solution, type and amount of solutes dissolved in the solution, pH of the solution, volume of solution, etc.) to construct a concentration curve that includes concentration of the known analyte on one axis, and a characteristic of the spectrum on the other axis (e.g., the amplitude of the largest peak, the total area under the curve, etc.).

In some non-limiting examples, the block 622 can include a computing device determining a speed of sound of a surface protein of the analyte (e.g., a capsid protein), based on the determined resonant frequency. For example, if a computing device has received a size of the analyte (e.g., a width, a diameter, a radius, etc.), the diameter of the analyte can be divided by the resonant frequency of the spectrum (e.g., by the computing device) to determine the speed of sound of the surface protein. In alternative cases, if a computing device has received the speed of sound of the surface protein of the analyte, the resonant frequency can be multiplied by the speed of sound of the surface protein (e.g., by the computing device) to determine the size of the analyte (e.g., the diameter of the analyte). In some cases, a computing device can store the speed of sound, and size of the analyte.

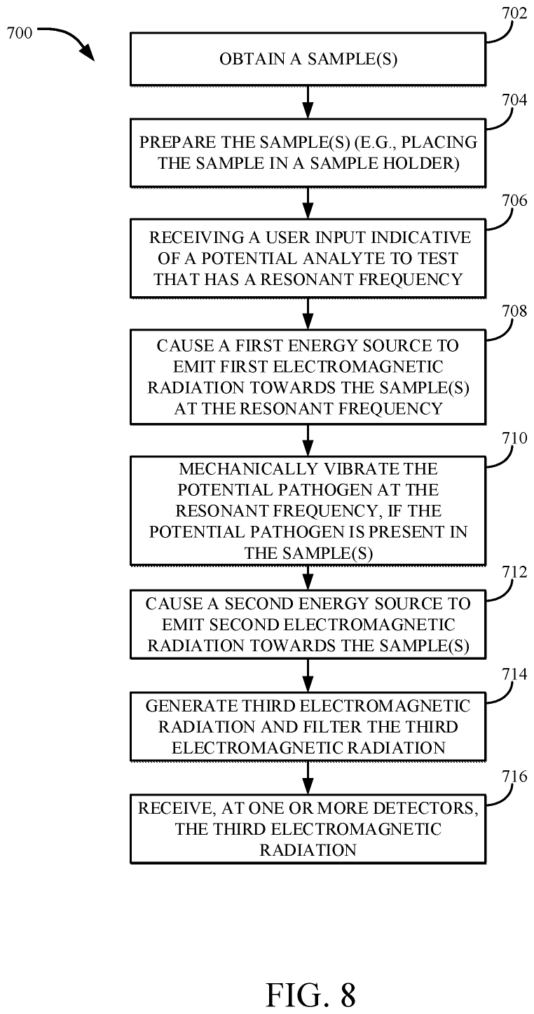

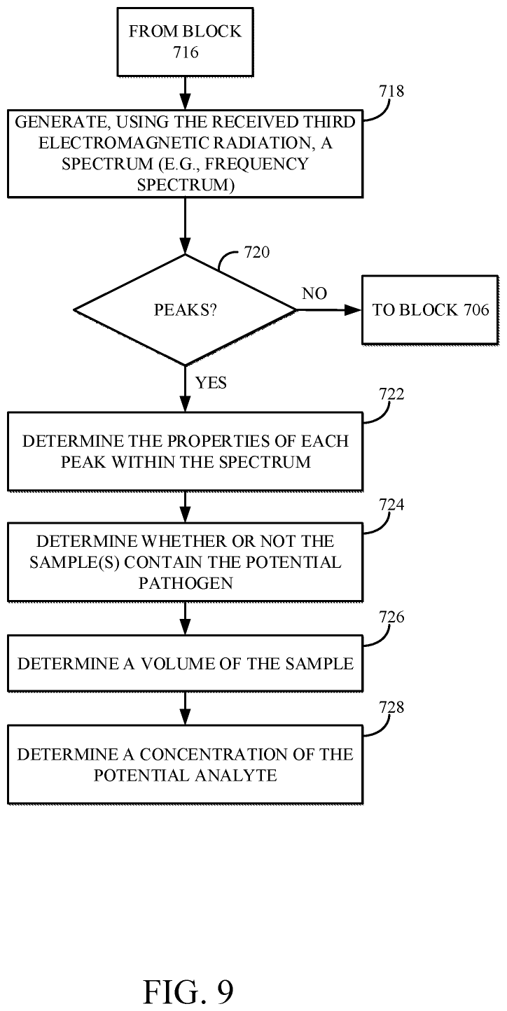

FIGS. 8 and 9 show a flowchart of a process 700 for detecting an analyte within a sample. This process can be implemented using the previously described systems (e.g., the system 100). In addition, some or all of the blocks of the process 700 can be implemented using one or more computing devices (e.g., the computing device 112), as appropriate. In some cases, the analyte can be a biological analyte including a virus, a bacteria, a fungi, a eukaryote, etc.

At 702, the process 700 can include obtaining a sample, which can be the same as the block 602 of the process 600. In some non-limiting examples, this can include obtaining multiple samples, each from a different individual. In this case, each of the samples (e.g., swabs) can be submerged (or loaded) into respective containers that each have their own solution, or can be submerged (or loaded) into a common container filled with a solution. In this case, a computing device can receive information regarding the source of each sample (e.g., an identifier of each person), so that if the results indicate a potential pathogen does not present in each sample container (or common sample container) each of the sources can be identified as being free of the potential pathogen. If however, the results indicate that the potential pathogen exists within one of the sample containers or the common sample container, then the source of each sample can be identified as potentially having the particular pathogen. This multiple sample approach, stated another way, is similar to pool testing. In this way, multiple samples each from a different source are pooled into a single sample holder (or multiple sample holders processed at the same time). In some cases, each sample having its own respective sample holder can be advantageous in that, if the results from testing multiple different samples indicate that the potential pathogen is in one or more of the multiple sample holders, then individual sample holders having a respective sample can simply be processed again (e.g., because no sample is “used up” during testing).

In some non-limiting examples, the sample can be a fluid sample (e.g., a gas sample). In this case, for example, the sample does not need to be prepared, as the testing can be completed on the sample. In other words, the sample can be the volume of a fluid at a point in time in which the fluid (e.g., gas containing particulates, aerosols, etc.) passes by though the energy source(s) and detector(s) (e.g., of the system 100). In other configurations, for example, the sample can be just the interior volume of the housing of the system to detect the analyte (e.g., the system 100). In these cases, for example, the system that can implement the process 700 can be integrated within an HVAC system, an air purifying system, a water supply system, a vent within a room of a building, a duct, etc.

At 704, the process 700 can include preparing a sample(s) (e.g., placing the sample within a sample holder), which can be the same as the block 604 of the process 600. In some cases, for the pooling approach, multiple samples each from a different source can be combined into a single sample holder, or can each be placed into a respective sample holder. Regardless, the one or more sample holders can be loaded into a housing of a system for detecting an analyte (e.g., the system 100).

At 706, the process 700 can include a computing device receiving a user input indicative of a potential analyte (e.g., a potential pathogen) to test that has a resonant frequency. In some cases, this can include a computing device receiving a user input indicating a particular analyte for testing of the sample. For example, a computing device can receive a user input indicating a type of virus to test. In some cases, the block 706 of the process 700 can include a computing device determining the resonant frequency for the potential analyte, based on the received user input. For example, a computing device, after receiving the user input, can query a database that includes resonant frequencies for each analyte to determine the resonant frequency. In some cases, the database can be built using previously determined resonant frequencies for samples having the respective analyte (e.g., using the process 600). In other cases, a computing device can determine the resonant frequency based on a size of the analyte. For example, a computing device can determine the resonant frequency based on a predetermined size of the analyte by comparing the predetermined size to a graph (or data) that includes a function of the resonant frequency for various diameter sizes of analytes. In some cases, the block 706 of the process 700 can include a computing device receiving a user input indicative of the resonant frequency. For example, a computing device can receive a frequency value (e.g., inputted by a user).

At 708, the process 700 can include a computing device causing a first energy source to emit first electromagnetic radiation towards the sample at the determined (or received) resonant frequency. The block 708 of the process 700 can be the same as the block 606 of the process 600. For example, the first electromagnetic radiation can be a frequency sweep over a frequency range that includes the resonant frequency (e.g., determined or received at the block 706).

In some non-limiting examples, the first energy source can be unrelated to the determined (or received) resonant frequency. In this way, for example, the first energy source can include light emissions at different frequencies corresponding to many resonant frequencies of different analytes (e.g., pathogens, such as a pathogen list).

At 710, the process 700 can include mechanically vibrating the potential pathogen at the resonant frequency, if the potential pathogen is in the. For example, if the potential pathogen is present within the sample, then the first electromagnetic radiation will force the potential pathogen to mechanically vibrate at the resonant frequency (and resonantly vibrate at harmonic frequencies of this resonant frequency that is a fundamental frequency). However, if the potential pathogen is not present within the sample, then the first electromagnetic radiation will not force a different pathogen to mechanically vibrate at the resonant frequency.

At 712, the process 700 can include a computing device causing a second energy source to emit second electromagnetic radiation towards the sample, which can be the same as the block 608 of the process 600.

At 714, the process 700 can include generating third electromagnetic radiation, which can be the same as the block 610 of the process 600. In some cases, if the analyte within the sample resonantly vibrates at its resonant frequency (e.g., due to the first electromagnetic radiation), then the interaction between the resonantly vibrating analyte and the second electromagnetic radiation will generate the third electromagnetic radiation. However, if the analyte within the sample does not resonantly vibrate at its resonant frequency (e.g., due to the first electromagnetic radiation), then the third electromagnetic radiation will not be generated.

In some non-limiting examples, at the block 714, the process 700 can include a computing device filtering the third electromagnetic radiation (e.g., before reaching a detector), which can be the same as the filtering of the block 610 of the process 600.

At 716, the process 700 can include a computing device receiving, at one or more detectors, the third electromagnetic radiation, which can be the same as the block 612 of the process 600.

At 718, the process 700 can include a computing device generating, using the received third electromagnetic radiation, a spectrum, which can be the same as the block 614 of the process 600. In some non-limiting examples, the process 700 can include a computing device combining (e.g., adding, averaging, etc.) multiple spectrums, acquired from different iterations of the blocks 708-716 of the process 700. In this way, multiple testing runs of the sample for the potential analyte can be completed, and combined to, for example, increase the statistical power of the testing.

At 720, the process 700 can include a computing device determining whether or not any peaks are present within the spectrum, which can be the same as the block 616 of the process 600. For example, if at the block 720, a computing device determines that no peaks are present within the sample (e.g., that are greater in amplitude than a threshold value), the process 700 can proceed to the block 706. In this case, a different resonant frequency can be selected, which can correspond to a different pathogen, and a computing device can then cause the first energy source to emit another first electromagnetic radiation at the different resonant frequency. If, however, at the block 720, a computing device determines that one or more peaks are present within the spectrum, the process 700 can proceed to the block 722.

At the block 722, the process 700 can include a computing device determining the properties of each peak within the spectrum, which can be the same as the block 618 of the process 600. For example, this can include a computing device determining the frequency and amplitude of each peak within the spectrum.

At the block 724, the process 700 can include a computing device determining whether or not the sample contains the potential pathogen. In some cases, if at the block 720, one or more peaks were determined, then the mere presence of the peaks indicates to the computing device that the sample (or multiple samples) contains at least some of the potential pathogen (e.g., assuming non-overlap of the first electromagnetic radiation with resonant frequencies of other pathogens present in the sample). In other cases, a computing device can determine whether or not the sample (or multiple samples) contains the potential pathogen based on the frequency a determined frequency peak within the spectrum and the frequency of the second electromagnetic radiation. For example, if a computing device determines that the difference between the frequency of a peak of the spectrum and the frequency of the second electromagnetic radiation results in the resonant frequency of the potential analyte (or an integer multiple thereof), then the computing device determines that the analyte is present within the sample (or multiple samples). In some cases, a computing device can determine that the sample (or multiples samples) does not have the potential analyte based on no peaks being present within the spectrum, or no peaks being within the spectrum with a frequency that is separated from the frequency of the second electromagnetic radiation by an integer multiple of the resonant frequency.

Ins some non-limiting examples, in the multiple sample approach, if at the block 722 a computing device determines that the potential pathogen is not present within the multiple samples (e.g., a single sample holder containing multiple samples from different sources, or multiple sample holders each containing a sample from a different source), then the computing device can determine that all the different sources associated with the multiple samples do not have the potential pathogen. Conversely, if at the block 722 a computing device determines that the potential pathogen is present within the multiple samples, then a computing device can determine that all the different source associated with the multiple samples potentially have the potential pathogen. In some cases, a computing device can generate a report of whether or not the multiple samples (or sample) contains the potential pathogen.

In some non-limiting examples, if a computing devices determines that the potential pathogen is present within the multiple samples, then a computing device can instruct (e.g., a user by presenting a display) that each of the individual samples (assuming each sample is contained in a respective sample holder) it tested again. Thus, the process 700 (e.g., blocks 708-728) can be completed for each individual sample.

In some non-limiting examples, the block 724 can include a computing device determining an amount of overlap between the spectrum and a previously acquired spectrum of the potential analyte. Then, a computing device can determine whether or not the amount of overlap exceeds a threshold (e.g., is greater 50 percent overlap). If the computing device determines that the overlap exceeds the threshold the computing device can determine that the sample includes the potential analyte. If, however, the computing device determines that the overlap does not exceed the threshold, the computing device can determine that the sample does not include the potential analyte. In this case, for example, a computing device can implement this process to compare the spectrum to other previously acquired spectrums each of a different analyte to determine if the spectrum matches with any other spectrums for different analytes.

In some non-limiting example, the block 724 can include validating that the potential analyte is present within the sample. In some cases, this can include testing the sample with a different test for the potential analyte including a PCR reaction, an antibody test, or an antigen test to validate that the potential analyte is present within the sample.

At 726, the process 700 can include a computing device determining a volume of the sample. In some cases, the volume can be a default volume (e.g., for a standard sample volume), while in other cases, a computing device can receive a user input indicating the volume of the sample.

At 728, the process 700 can include a computing device determining a concentration of the potential analyte, based on the third electromagnetic radiation (e.g., the spectrum). For example, a computing device can compare each amplitude of each peak and the determined volume of the sample to a calibration curve to determine the concentration of the potential pathogen in the sample. As another example, a computing device can select the peak with the highest amplitude and can compare this peak (and the volume of the sample) to a concentration curve to determine the concentration of the analyte within the sample. In some cases, the concentration curve can be a standard concentration curve, or can be specific to the potential analyte. For example, the concentration curve can be previously created based on a serial dilution of the potential analyte (e.g., from the process 600).

The sample of the methods 200, 300, 400, 500, 600, 700 can include and/or be selected from the group consisting of saliva, sputum, blood, breath exhalant, a nasopharyngeal swab, an oropharyngeal swab, stool, and a surface swab. The methods 200, 300, 400, 500, 600, 700 including a sample can be acquired from a human subject.

### EXAMPLES

The following examples have been presented in order to further illustrate aspects of the disclosure, and are not meant to limit the scope of the disclosure in any way. The examples below are intended to be examples of the present disclosure and these (and other aspects of the disclosure) are not to be bounded by theory.

The following examples set forth, in detail, experiments for detecting COVID-19 by applying the systems and methods described herein. The following examples are presented by way of illustration and is not meant to be limiting in any way.

### Example 1

In one non-limiting example, the sensitivity and specificity of the above-described photoacoustic spectroscopy techniques of the present disclosure are applied to live SARS-CoV-2.

Different energies have been used for a long time to vibrate materials at a resonant frequency. For example, magnetic resonance imaging (“MM”) is fundamentally built upon the dipole nature of hydrogen and the ability to use magnetic fields and radio frequency (“RF”) energy to study various bodies (e.g., humans) that include hydrogen (or even other atoms). As another example, some have used photon absorption or acoustic energy to excite particles. While inducing resonant vibrations can be achieved using a wide-variety of energies applied to a wide-variety of materials (e.g., from organic molecules to nanocrystals), the utility of these induced vibrations has generally not yielded suitable results (e.g., on par with MRI, or the like).

Spherical particles oscillate with different modes described by Lamb's theory, which classifies spherical particles into two categories: spheroidal and torsional modes. These modes are labeled by the angular momentum quantum number (l), where l=0, 1, 2, . . . for spheroidal modes, and l=1, 2, 3, . . . for torsional modes. The sequence of eigenmodes in increasing order of energy is indexed by n=1, 2, 3, . . . Thus, Lamb's theory predicts that the frequencies of the eigenmodes (n, l) scale over the diameter of the sphere (d).

Further interrogation of a particle during resonance is complicated by a variety of known phenomenon, such as Brillouin and Raman scattering. Though different in nature, the terms “Brillouin” and “Raman” are sometimes used interchangeably in the literature. Some researchers have posited that l=1 is not Raman (or Brillouin) active. Thus, some have used microwaves to induce the l=1 mode of spherical particles in the hope of being able to study or manipulate the particle without the confounding nature of Raman or Brillouin interactions.

As further complications, researchers studying methods for treating pathogens have previously demonstrated that microwaves can be used to couple to the pathogens inducing confined acoustic vibrations sufficiently strong to rupture the microorganisms. Specifically, some have demonstrated that at least some coronaviruses can be ruptured by inducing acoustic vibrations under physiological conditions of salinity and acidity. Similarly, measurements of the resonance phenomena of the H3N2 and H1N1 viruses have shown high extinction rates at the microwave frequency near 8 GHz.

Systems and methods provided herein, advantageously, need not focus on careful selection of angular momentum quantum number (1) or protect against induced, undesired rupture of samples.

### Experimental Design

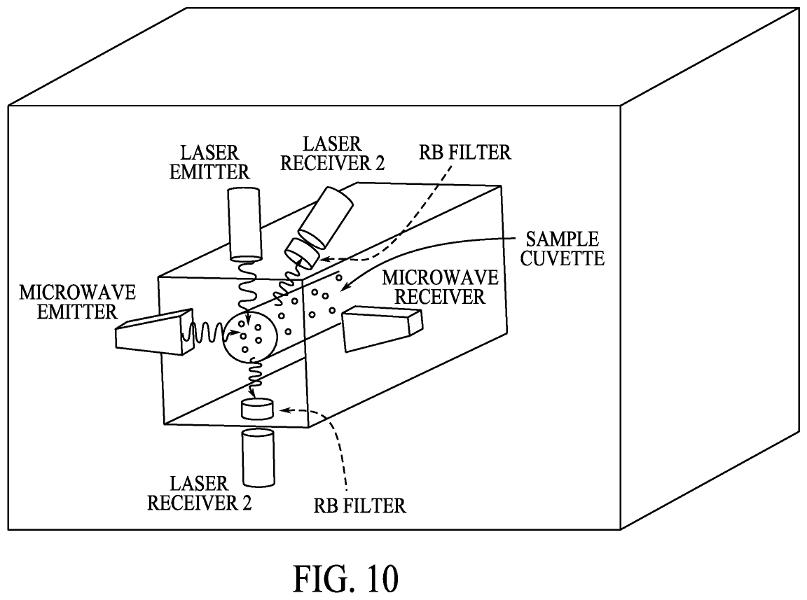

Turning to FIG. 10, a schematic diagram is provided setting out an experimental detection system. The detection system includes (1) a portable instrument composed of a microwave transmitter, a laser, a sample holder, detectors, control electronics and a data processing station using readily available components, and (2) a sample interface that allows the user to introduce samples in a variety of settings and formats (e.g. breath exhalant, saliva, etc.).

The system combines microwave and laser spectroscopy into a novel biosensing system to rapidly detect SARS-CoV-2 virus (<10 seconds) at very high sensitivity (<5 virus/cc at 99.99% detection probability) and specificity. The technique is ‘label-free’, does not necessarily require sample pretreatment, and uses inexpensive consumables safe for use in both laboratory and point-of-care settings. Importantly, the system requires little to no sample manipulation by device operators rendering it suitable for wide deployment where its use can facilitate resumption of economic activity.

The system is designed to handle trace samples as either aerosols (in a cuvette) or on a substrate such as a swab. The viral particles are embedded in organic material at some unknown concentration. Both the microwave (about 1-50 GHz) and optical (780 nm) illumination wavelengths are chosen to easily penetrate organic material in order to probe the embedded virus particle. Detection system equipment includes three primary components: (1) a tunable microwave source with frequency range covering the expected coronavirus resonance frequencies (0-40 GHz), such as the Keysight M8195A-002 Arbitrary Waveform Generator. (2) An interrogation laser (e.g. the New Focus TLB-6712) that operates at 780 nm to optimize detection efficiency and ensure optical penetration. It has a narrow optical linewidth GHz) relative to the coronavirus resonance frequency. The laser is tuned to the Rb resonance so that the elastically scattered laser light can be filtered out using a Rb gas cell (e.g. Thorlabs GC19075-RB). (3) A high-speed, high-quantum-efficiency, single-photon-sensitive optical detector detects the scattered target photons (e.g. Hamamatsu H7421). An optical spectrum analyzer is used to analyze the full spectrum of the scattered light (e.g. Yokogawa AQ6370D). Optical detection occurs in a shot-noise limited regime (only light directly related to target viruses is received due to the filtering scheme of the detection system) enabling the highest detection sensitivity. Photon signals are captured via data acquisition hardware and a computer system.

Size and power requirements: the developed system is the size of a microwave oven and operates on conventional 110V AC power. Samples are inserted into the system in optical glass cuvettes and sample analysis is performed in an analysis chamber. Importantly, little to no sample handling is necessary for sample analysis, and the sample cuvette can remain sealed throughout. The system is readily operable by a user after brief training. Importantly, the machine operator does not necessarily need specialized specimen handling training and the system does not require placement in a restrictive biosafety environment.

Consumables and disposables: the experimental detection system is engineered to use conventional consumables. Such consumables are widely available and are made by multiple US-based manufacturers in large quantities at low cost for existing testing purposes.

Sample collection and analysis: The system is capable of processing multiple specimen types including exhaled breath, nasopharyngeal and oropharyngeal swabs, sputum, saliva, throat washings, and blood. Little to no specimen preparation is required. Initial experiments can focus on saliva samples, which contain higher numbers of SARS-COV-2 viral particles than nasopharyngeal swabs and have a simpler sample collection procedure. It should be noted that the detection system is readily adaptable to the interrogation of any sample type and, given anticipated high sensitivity, the system can be adapted to exhaled breath sampling, using Ultra-Low Particulate Air (“ULPA”) filters to concentrate expired aerosols.

Testing, calibrating and validating the system: A frozen (−80° C.) biospecimen bank of sputum and saliva samples, validated by PCR to contain SARS-COV-2, Influenza virus, and Respiratory syncytial virus (RSV) can be used. The sample bank of contains hundreds positive and negative SARS-COV-2 test samples. These four different sample groups of negative (control), SARS-COV-2+, Influenza+ and RSV+ can be thawed and used for testing and validation of the detection method and system.

Connectivity: the system can be designed to immediately provide the operator with a positive/negative result for the test. The system can provide a virus-specific readout. The system can also be equipped with middleware that connects the analyzer to Laboratory Information System (“LIS”) software. The LIS can subsequently interface with the Electronic Medical Records (“EMR”) and transfer the lab results in compliance with Health Level Seven International (“HL7”) standards. In this structure, the middleware enables messaging, automation, inspection preparedness/compliance, and quality control capabilities, and the LIS handles patient check-in, order entry, results entry, patient demographics, specimen processing, and reporting ability. A software module that provides connectivity with mobile telephone devices and cell phone networks to facilitate centralization of encrypted data with appropriate privacy protections for public health purposes can also be used.

Training: operators require minimal training to use the detection system. The system is a “push-button” system that does not require extensive training to operate and the results require minimal interpretation. Appropriate training can be required for use of personal protective equipment and collection of samples following standard protocols.

Radiation Safety concerns: advantageously, there are no special shielding requirements for operating the system. The design includes electromagnetic shielding to protect the operator from electromagnetic radiation.

Infection control: the detection system can be a closed system that does not confer infection risks on operators. Samples can be collected by individual patients or trained personnel, donning proper personal protective equipment (“PPE”), which includes masks, gloves, goggles, gown, head cover, shoe cover and access to hand sanitizer, soap and water following adequate infection control measures including hand hygiene and adequate biosafety precautions to protect individual and the environment.

Device disinfection: the small airtight interrogation chamber of the detection system can be made of optical glass and can be removed and disinfected, and a new interrogation chamber can be inserted into the device. The optical glass material of the interrogation chamber can allow various disinfection methods such as autoclave, immersion in 70% isopropyl alcohol solution, and introduction of hydrogen peroxide gas into the chamber. A used interrogation chamber can even be placed in a box and disinfectant solution can be sprayed on it. Sterilization of the system can be performed in accordance to FDA guidelines.

Deployment: the above-described system can be readily deployed to multiple locations, such as hospital waiting rooms, emergency departments, respiratory illness clinics, general inpatients floors, clinical laboratories, research labs, and airports. Use cases that would be directly relevant to broader social goals such as fit-for-work assessments are also practical. In these use cases, simplified operations manuals can be provided and the device can be deployed more broadly outside of healthcare contexts, to numerous locations including shopping centers, houses of worship, sporting events, and other public spaces including airports, schools, sport events, concerts, restaurants, and the like.

### CONCEPT DISCUSSION

The detection system relies on an induced viral vibration which produces a weak optical sideband in a laser probe beam. This optical sideband would ordinarily be considered undetectable because of the large amount of unwanted light scatter. The envisioned system and method addresses this issue by selectively filtering out unwanted light using a laser frequency matched to the narrow atomic absorption line of a rubidium vapor cell, which can filter unmodulated and unwanted background light. This enables extraordinarily sensitive photodetection of the weak signal from the vibrating virus. The envisioned system can selectively vary the microwave frequency, which will enable selective detection and classification of viral particles by matching detected signals to known viral vibration spectra. In this manner, the envisioned detector will serve not only to detect and classify SAR-CoV2 with sensitivity unmatched by existing alternative technologies but could also be employed as a rapid diagnostic method in future viral pandemics.

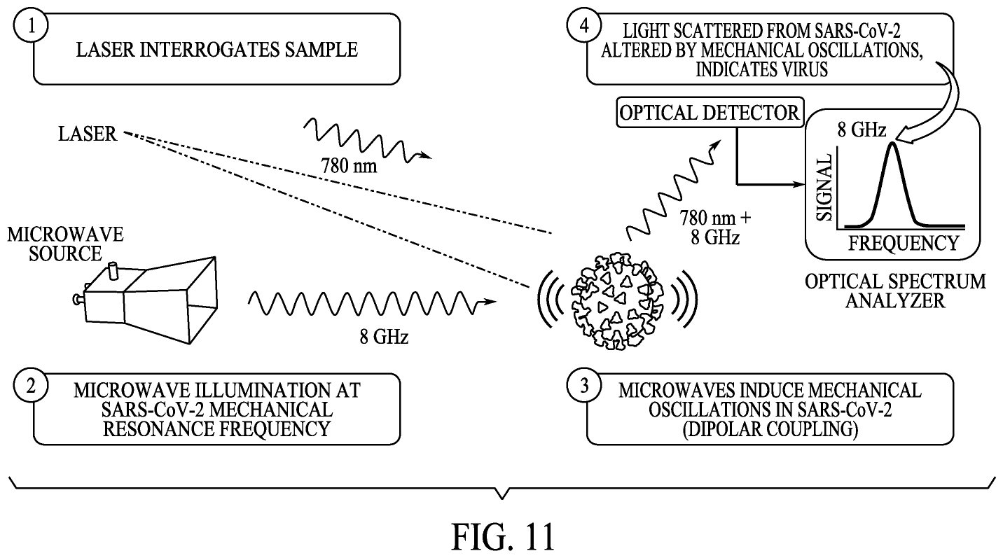

Without being bound by theory, in the experimental detection system concept, microwave radiation is used to resonantly excite mechanical vibrations in the SARS-CoV-2 virus at frequencies determined by the parameter fᵥ=dᵥ/v_{c} where dᵥ is the virus diameter and v_{c} effective speed of sound of the capsid protein surrounding the virus. The physics behind this process is illustrated in FIG. 11 and is additionally described herein. A spectrally narrow laser illuminates the sample volume and is scattered by the vibrating virus and is collected by a low-noise optical detector. The vibration modulates the phase and broadens the optical spectrum of the scattered light. The spectrally broadened scattered light is easily distinguished from light that is scattered from other unmodulated sources such as dust, aerosols and contaminants and is filtered using a novel optical filter that efficiently blocks unmodulated laser light from reaching the detector. Presence or absence of this frequency-shifted light is used to establish the presence or absence of the virus. In addition to indicating the presence of the virus, the scattered light also contains information that are specific to target materials (such as the size, charge, mass, etc.) and can be used to both detect and classify the virus.

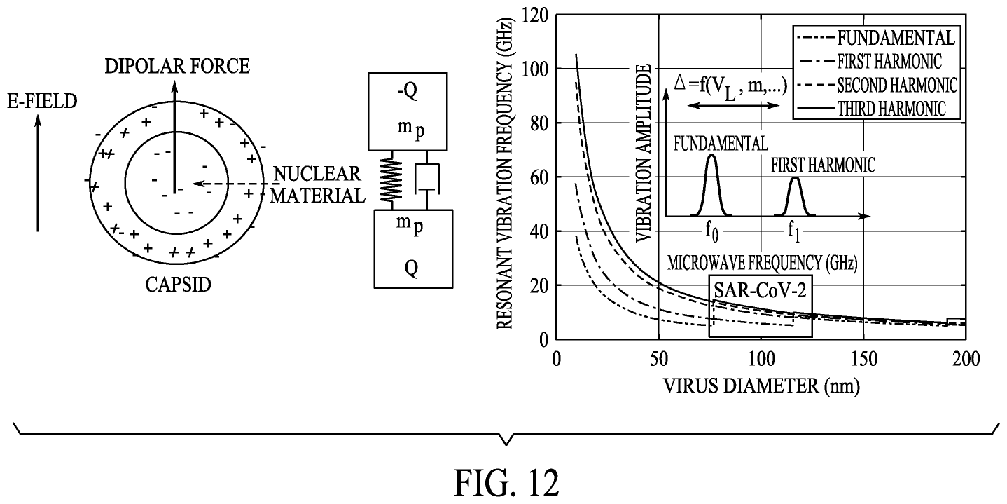

Microwave Excitation of Confined Acoustic Vibration: prior work has demonstrated microwaves efficiently couple to coronaviruses inducing confined acoustic vibrations sufficiently strong to rupture the virus under physiological conditions of salinity and acidity. Measurements of the resonance phenomena of the H3N2 and H1N1 viruses have previously shown high extinction rate at the microwave frequency near 8 GHz. Human coronaviruses, and in particular SARS-CoV-2, are similar in size and shape (roughly 80-120 nm) to H3N2/H1N1. Consequently, for SARS-CoV-2, it is expected that the nominal resonant frequency is to be about 8 GHz. However, due to natural variation in the size and elastic properties of the virus, it is expected that this frequency will vary by 10-20%. Additionally, analysis suggests that detection performance can improve with the addition of 2-3 harmonics of the fundamental frequency. For these reasons, the microwave frequency is swept from about 5-15 GHz to capture this variation. Without being bound by theory, the physical process that leads to this behavior is the separation of electrical charge between the genomic material in the core and oppositely charged proteins in the capsid. The dipolar field from this charge separation couples efficiently when the frequency of the oscillating electromagnetic field matches the particle's mechanical resonance frequency and leads to strong mechanical oscillations of the virus at the same frequency. This interaction has been previously modeled in the literature as a charged damped mass-spring system driven into oscillation by the force imparted by the electromagnetic field as shown in FIG. 12. In FIG. 12, the left most depiction shows the electric field generated in a particle by an incident microwave, which induces a dipolar force on the particle. This electric field is similar to the mechanical spring-mass-dashpot model shown in the center depiction. The mechanical frequency depends on the capsid size and is used as a viral classifier. The oscillation frequency is related primarily to its shape, size, stiffness, mass density and the speed of sound of the protein comprising the capsid. The resonance frequency shown in FIG. 12 can be estimated using Lamb's theory.

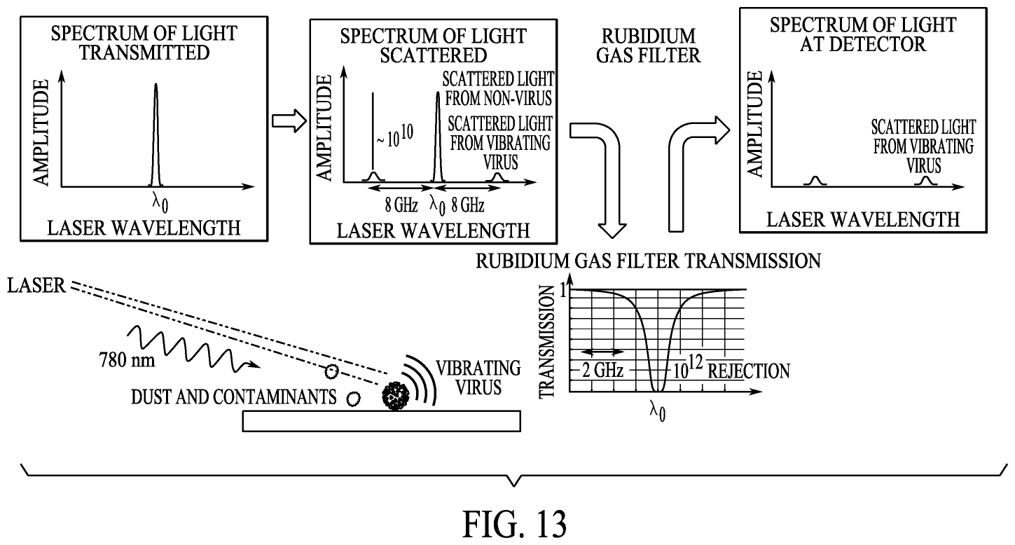

Optical Detection of Weak Frequency-Shifted Light: the interaction of optical radiation with the virus is illustrated in FIG. 13. A stable narrow 780 nm laser precisely tuned to the rubidium absorption wavelength illuminates a ˜1 cm³ sample volume. Light interacting with the vibrating virus is Doppler shifted by the vibration frequency and appears as a sideband to the unshifted laser light. Since the size of the virus is small compared to the illuminated area, the amount of frequency-shifted scattered light is small. Detecting this weak light is challenging since it is often 10 or more orders of magnitude smaller than other sources of scattered and reflected light from the sample (e.g. aerosols, dust, contaminants and solid surfaces) that do not interact with the microwave. It can be important to block this unwanted light in order to detect the weak signal from the virus. This approach is to use a laser tuned to the absorption line of rubidium and to use a rubidium cell in front of the detector as an absorption filter. A suitably designed rubidium vapor cell can have absorption linewidths as narrow as 500 MHz in width and an optical depth exceeding 10-12 orders of magnitude at resonance. Light that retains the spectrum of the transmitted light (e.g. light scattered by dust, aerosols, etc.) is effectively blocked from reaching the detector. However, light from a vibrating virus appears as a spectral sideband (>5 GHz) that is outside the rejection band of the filter and therefore is not blocked from reaching the detector.

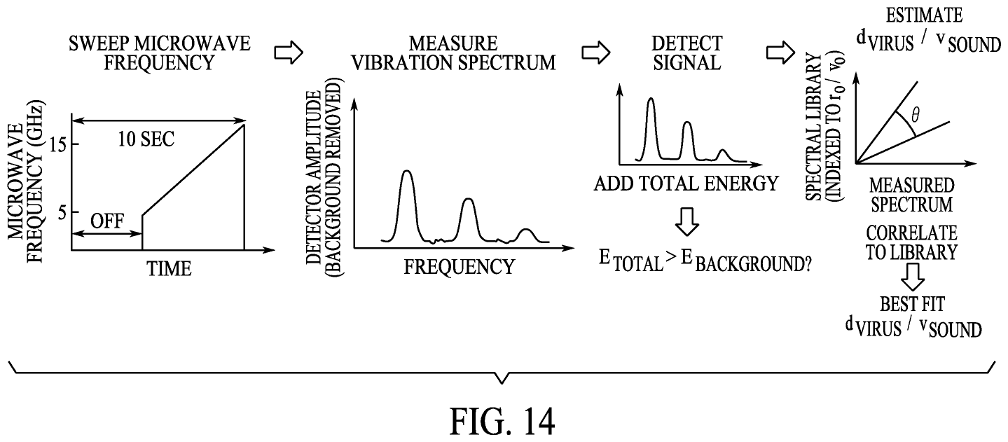

Signal Detection and Processing: the signal processing chain of the envisioned system is shown in FIG. 14. Initially the sample is illuminated by an optical laser with microwave power off to measure background light. The microwave power is then turned on and linearly swept from 5 to 15 GHz over roughly 10 seconds in steps of roughly 1 GHz. Time-varying photodetector output, which is proportional to the microwave absorption spectrum of the virus, is collected by a data recording and processing system. The time-integrated signal strength is compared to background and a detection threshold set based on desired sensitivity and false alarm rate for the system. To determine the type of virus detected, two approaches are envisioned. One approach is to build a spectral database collected under varying conditions using the instrument and to use this to match spectra collected in the field. A second approach is to apply Lamb theory to construct a filter bank against which measured spectra are matched (using standard methods such as spectral angle mapper) and from which the parameter fᵥ=dᵥ/v_{c} is estimated. This parameter is then compared against approximate values of dᵥ and v_{c} which are known typically to +/−20% for known viruses.

### Example System Performance Model

A system designed as described above has been modeled to have a performance using currently available data on the coronavirus and other related viruses. This model shows that the envisioned approach has a sensitivity orders of magnitude higher and testing time significantly shorter than current laboratory methods.

System Performance: a model was constructed to estimate overall system sensitivity. Microwave absorption is modeled using the formalism summarized in Table 1. In general, the resonance frequency fᵥ is derived from Lamb's theory and is a function of dᵥ (see FIG. 12). The typical microwave intensity required to achieve such a displacement is approximately 200 W/m² which can be achieved with low-cost low-power RF components. The optical scattering model consists of two components. First is the scattering cross section. This is estimated by assuming Rayleigh scattering, which is reasonable for particles that are much smaller than the optical wavelength. Second, the vibration is assumed to induce a phase modulation on the scattered beam which appear as sidebands to the carrier frequency whose amplitude is given by

$${n}_{vib} = {\vert {J}_{1} ( \frac{4 π}{λ} {x}_{max} ) \vert}^{2} = 1.6 \times 1 {0}^{- 5}$$

where J₁(x) is the Bessel function of order one. The principal equations and parameters are summarized in Table 1.

TABLE 1
Principal system parameters for RF and optical processes.

| Principal RF System Parameters | | Principal Optical System Parameters | |
|---|---|---|---|
| ${σ}_{α bs} = \frac{{Qq}^{2}}{{ɛ}_{v} {m}_{v} {ω}_{0}}$ | 3 × 10⁻¹³ m² (per virus) | ${σ}_{opt} = \frac{2 {π}^{5} {d}_{v}^{6} {n}_{med}^{4}}{3 {λ}^{4}} {\vert \frac{{( \frac{{n}_{v}}{{n}_{med}} )}^{2} - 1}{{( \frac{{n}_{v}}{{n}_{med}} )}^{2} + 2} \vert}^{2}$ | 9 × 10⁻¹⁸ m² (per virus) |
| ${x}_{max} = 0.6 {P}_{max} \frac{π {d}_{v}^{2}}{8 {m}_{v} {ω}_{0}^{2}}$ | 0.27 nm | ${η}_{vib} = {\vert {\int }_{1} ( \frac{4 π}{λ} ) {x}_{max} \vert}^{2}$ | 4.7 × 10⁻⁶ |
| ${P}_{max} = \frac{14 Q {qE}_{0}}{π {d}_{v}^{2}}$ | 0.26 MPascal | $SNR = {N}_{virus} {η}_{sys} \frac{{P}_{laser} {T}_{eff}}{hv π f \text{/} {\#}^{2}} \frac{{σ}_{opt}}{{A}_{spot}} {η}_{vib}$ | 3/virus |

mᵥ = 20 MDalton
w₀ = 2π × 8 GHz
εᵥ = 64 × 8.854 × 10⁻¹² F/m
q = 1.4 × 10⁶C
dᵥ = 100 nm
Q = 2
λ = 780
n_{med} = 1.33.
nᵥ = 1.48
Pₗₐₛₑᵣ = 150 mW
T_{eff} = 5 sec
hv = 3 × 10⁻¹⁹J
Aₛₚₒₜ = 1 cm²
f/# = 0.4

Estimated System Sensitivity: sensitivity of the system is estimated assuming Poisson detection statistics. This is justified due to the negligible background noise (only scattered light from vibrating virus falls on the detector) and negligible detector noise (dark current and read noise are expected to be <0.1 e/pix in a 10 second integration time). Setting the detection threshold at 1 count leads to a false alarm probability P_{fa}<5×10⁻³, which is well below typical false negative rates of commercial systems for virus detection. Table 2 summarizes system sensitivity as a function of virus size. Smaller viruses scatter smaller amounts of light (see Table 1) and consequently the limit of detection is correspondingly higher. SARS-CoV-2 is a relatively large virus and its signature is predicted to have excellent detection performance with near single-virus/mL system sensitivity.

TABLE 2
Predicted system sensitivity for Poisson detection.

| Virus Size | Sensitivity | | |
|---|---|---|---|
| (nm) | 90% | 95% | 99% |
| 20 | 58000 | 70000 | 95000 |
| | (virus/mL) | (virus/mL) | (virus/mL) |
| 40 | 900 | 1100 | 1500 |
| 60 | 80 | 93 | 130 |
| 80 | 14 | 17 | 23 |
| 100 | 3.5 | 4.5 | 6 |
| 120 | 1.25 | 1.5 | 2 |

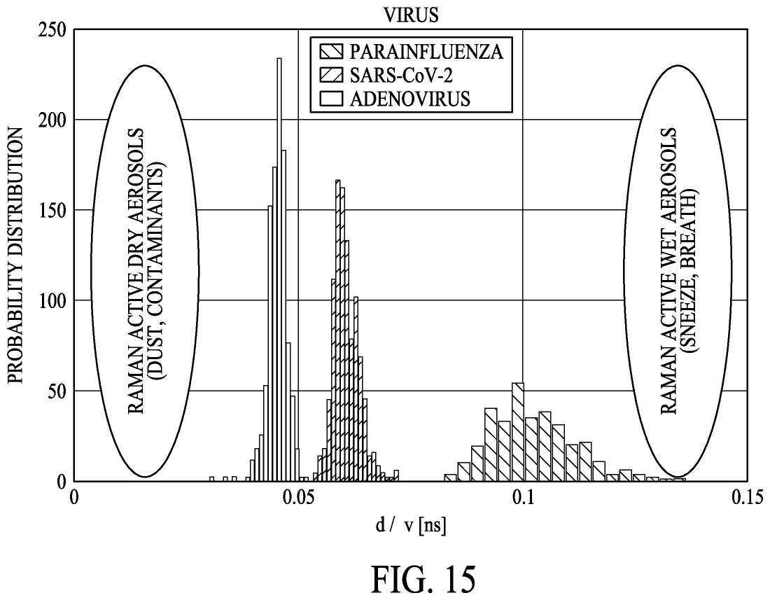

Estimated System Specificity: As described, the system can detect particles mechanically resonating at a specific frequency in response to microwave irradiation. Only particles of similar size, shape, sound speed, and charge distribution to SARS-CoV-2 will resonate at this frequency. Few naturally occurring materials meet these criteria. For those that do resonate (predominantly metals and spherically charge dielectrics), the parameter fᵥ=dᵥ/v_{c} is an estimated parameter from the processing chain (see FIG. 15). This parameter can be used as discriminant/classifier. Table 3 shows pathogens and corresponding fᵥ values of interest.

TABLE 3
Predicted system specificity for d/v classifier assuming
v_{c} = 1800 m/s ± 200 m/s. FDA
Recommended List of Virus for Wet Testing.

| Virus # | Virus | dᵥ (nm) | σᵥ (nm) | dᵥ/v_{c} | σ_{d/v} |
|---|---|---|---|---|---|
| 1 | Enterovirus | 27.5 | 1.25 | 0.015 | 0.002 |
| 2 | Rhinovirus | 30 | 1 | 0.017 | 0.002 |
| 3 | Adenovirus | 80 | 5 | 0.044 | 0.005 |
| 4 | SARS-CoV | 85 | 2.5 | 0.047 | 0.004 |
| 5 | SAR-CoV-2 | 95 | 15 | 0.053 | 0.011 |
| 6 | Influenza | 100 | 10 | 0.056 | 0.009 |
| 7 | MERS-coronavirus | 127 | 4.5 | 0.071 | 0.007 |
| 8 | Human coronavirus | 140 | 10 | 0.078 | 0.010 |
| 9 | Parainfluenza | 175 | 12.5 | 0.097 | 0.013 |
| 10 | Respiratory syncytial virus | 200 | 25 | 0.111 | 0.020 |
| 11 | Human Metapneumovirus | 375 | 112.5 | 0.208 | 0.075 |

It is noteworthy that the detector is insensitive to the common cold Rhinovirus (note false positive=0). Typical solid-state aerosols have much higher sound speed and will have much higher resonant frequencies and no signature in the 2-15 GHz range. The most likely false-positive candidates are other coronaviruses and perhaps some influenza viruses, causing false positives but still indicating the presence of an infectious virus. It is conceivable these viruses could be distinguished from SAR-CoV2 via differences in nuclear mass and charge. However, they could also be resolved via other tests. It is, however, important in that it demonstrates the utility and importance of the proposed system not only for the current pandemic but also in the future.

Table 4 provides a comparison of the above-described, microwave induced resonance for assaying coronavirus by light emission (“MIRACLE”) system.

TABLE 5
Comparison of the MIRACLE system with current alternative diagnostic methods.

| | | | | Limit of | | | | Consumable |
|---|---|---|---|---|---|---|---|---|
| Technology | Approach | Class | Sample | detection | Time to Result | | Throughput | Cost/Test |
| MIRACLE | Virus | Lab/POC | Breath, | <5 viral | 1-10 s (90 × | | +++++++++ | $1-2 |
| | | | Nasopharyngeal | particles | faster) | | | (estimated |
| | | | (NP) swab, | per mL | | | | ~20 × lower |
| | | | sputum, stool, | (~1,000 × | | | | cost) |
| | | | saliva, blood, | more | | | | |
| | | | breath exhalate | sensitive) | | | | |
| | | | (sample agnostic) | | | | | |
| PCR | Viral | Lab | NP swab, | 10 | 2-8 | h | +++ | Comparative |
| | RNA | | sputum, stool | copies/μL | | | | cost data |
| PCR-POC | Viral | POC | NP swab, | 10 | <1 | h | +++ | across the |
| | RNA | | sputum, stool | copies/μL | | | | rapidly |
| ddPCR | Viral | Lab | NP swab, | 0.02-0.1 | 2-4 | h | +++ | evolving |
| | RNA | | sputum, stool | copies/μ | | | | testing |
| NEAR | Viral | Lab/POC | NP swab, | 0.125 | 15 | min | + | landscape is |
| | RNA | | sputum, stool | copies/μL | | | | limited. A |
| LAMP | Viral | Lab/POC | NP swab, | 4 | 1 | h | ++ | typical |
| (OMEGA) | RNA | | sputum, stool | copies/μL | | | | consumable |
| RCA | Viral | Lab/POC | NP swab, | 100 | 2 | h | ++ | cost of $40 |
| | RNA | | sputum, stool | copies | | | | estimated by |
| SHERLOCK | Viral | Lab | NP swab, | 10-100 | 1.5 | h | + | Dr. Rosenberg, |
| | RNA | | sputum, stool | copies/μL | | | | who is |
| DETECTR | Viral | Lab | NP swab, | 10 | 1 | h | + | responsible |
| | RNA | | sputum, stool | copies/μL | | | | for SAR-CoV2 |
| NGS | Viral | Lab | NP swab, | <0.1 | Days | | +++ | testing |
| | RNA | | sputum, stool | copies/μL | | | | at MGH. |
| μNMR | Viral | Lab | NP swab, | 10 | 2 | h | +++ | |
| | RNA | | sputum, stool | copies/μL | | | | |
| LFA | IgG, IgM | POC | Blood | Variable | 15 | min | + | |
| ELISA | IgG, IgM | Lab | Blood | 100 pg/ml | 2-4 | h | +++ | |
| CLIA | IgG, IgM | Lab | Blood | | 30 | min | +++ | |
| EIA | IgG, IgM | Lab | Blood | | | | +++ | |
| ECLIA | IgG, IgM | Lab | Blood | | 20 | min | +++ | |
| VAT | Viral | Lab | NP swab, | 70 pg/ml | 4-5 | h | +++ | |
| | Protein | | sputum, stool | | | | | |
| ECS | IgG, | Lab/POC | Blood, NP | 5-50 pg/ml | 1 | h | ++ | |
| | Cytokine | | swab, sputum, | | | | | |
| | | | stool | | | | | |
| Microarrays | IgG types | Lab | Blood | 0.2-2 pg/ml | 1.5 | h | +++ | |
| IFM | Viral | Lab | Blood, NP | Variable | 3 | h | + | |
| | Protein | | swab, sputum, | | | | | |
| | | | stool | | | | | |
| WB | IgG, IgM, | Lab | Blood | >100 ng/ml | 4 | h | + | |
| | viral protein | | | | | | | |

Abbreviations CLIA — Chemiluminescence Immunoassay, ddPCR — Digital droplet PCR, DETECTR — DNA endonuclease-targeted CRISPR transreporter, ECLIA — Electrochemiluminescence immuinoassay, ECS — Electrochemical sensing, EIA — Enzyme Immunoassay, ELISA — Enzyme linked immunosorbent assay, LAMP — Loop Mediated Isothermal Amplification, POC — Point of care, RCA — rolling circle amplification, RPA — Recombinase Polymerase Amplification, SHERLOCK — specific high-sensitivity enzymatic reporter, VAT — Viral antigen test, WB — western blot, μNMR — Micro nuclear magnetic resonance, FIA — Fluorescence immunoassay, IFM — Immunofluorescence microscopy, Ig — Immunogloblin, LFA — Lateral flow assay, NEAR — Nicking endonuclease amplification reaction, NGS — Next generation sequencing, and PCR — Polymerase chain reaction.

Table: 5 Comparison of the MIRACLE system with current alternative diagnostic methods.

In order to ensure experimental success, various mitigation strategies are envisioned and provided below Table 6 for various unfavorable results.

TABLE 6
Envisioned risks and mitigation strategies.

| Risks | Impact | Mitigation strategy |
|---|---|---|
| Mechanical resonance for SARS- | Lower True Positive | Increase RF excitation strength. |
| CoV-2 is weaker than estimated | | |
| Resonance frequency of virus | Higher False Negative Higher False | Measure large number of samples |
| changes - Variability in virus media | Positive | in laboratory under varied |
| (pH, salinity, etc.) and/or | | conditions to measure distribution |
| interaction of virus with | | Vary excitation frequency to ensure |
| environment (drying, humidity, etc.) | | target frequency sampled. |
| Optical scattering efficiency is | Lower True Positive | Increase laser power, increase |
| lower than estimated | | signal integration time and/or |
| reduce laser spot size | | |
| Cross contamination from sample | Higher False Positive | Engineer air-tight chambers to |
| and operator handling | | prevent virus leakage. |
| | | UV treat sampling volume, |
| | | Require operators to don PPE. |
| Instrument specificity lower than | Higher False Positive | Collect multiple samples and repeat |
| expected from confounds - other | | measurement to eliminate artifacts, |
| (relatively) benign virus with | | |
| similar size OR other unexpected | | Follow up detection with |
| materials have same resonance | | confirmation sensor. |
| frequency | | |
| Parasitic light from non-viral | Lower True Positive | Use materials with low parasitic |
| material higher than expected | | light yield, increase laser power, |
| | | increase signal integration time |
| | | and/or reduce laser spot size |
| Unable to manufacture sufficient | Slower deployment time Higher | Buy components from multiple |
| instruments needed for utility- | system cost | vendors to ensure resilient supply |
| unable to find parts for volume | | chain |
| manufacturing; supply chain delays | | Minimize use of custom parts for |
| and disruption due to pandemic | | optics, lasers, detectors and |
| | | electronics |
| | | Seek governmental assistance for |
| | | priority access to parts |

The present disclosure has described one or more preferred non-limiting examples, and it should be appreciated that many equivalents, alternatives, variations, and modifications, aside from those expressly stated, are possible and within the scope of the invention.

It is to be understood that the invention is not limited to the particular non-limiting examples described. It is also to be understood that the terminology used herein is for the purpose of describing particular non-limiting examples only, and is not intended to be limiting. The scope of the present invention will be limited only by the claims. As used herein, the singular forms “a”, “an”, and “the” include plural non-limiting examples unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps can be combined with other elements, components, or steps that are not expressly referenced. Non-limiting examples referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

It is to be understood that the disclosure is not limited in its application to the details of construction and the arrangement of components set forth in the following description or illustrated in the following drawings. The disclosure is capable of other non-limiting examples and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein, unless otherwise limited or defined, discussion of particular directions is provided by example only, with regard to particular non-limiting examples or relevant illustrations. For example, discussion of “top,” “front,” or “back” features is generally intended as a description only of the orientation of such features relative to a reference frame of a particular example or illustration. Correspondingly, for example, a “top” feature can sometimes be disposed below a “bottom” feature (and so on), in some arrangements or non-limiting examples. Further, references to particular rotational or other movements (e.g., counterclockwise rotation) is generally intended as a description only of movement relative a reference frame of a particular example of illustration.

In some non-limiting examples, aspects of the disclosure, including computerized implementations of methods according to the disclosure, can be implemented as a system, method, apparatus, or article of manufacture using standard programming or engineering techniques to produce software, firmware, hardware, or any combination thereof to control a processor device (e.g., a serial or parallel general purpose or specialized processor chip, a single- or multi-core chip, a microprocessor, a field programmable gate array, any variety of combinations of a control unit, arithmetic logic unit, and processor register, and so on), a computer (e.g., a processor device operatively coupled to a memory), or another electronically operated controller to implement aspects detailed herein. Accordingly, for example, non-limiting examples of the disclosure can be implemented as a set of instructions, tangibly embodied on a non-transitory computer-readable media, such that a processor device can implement the instructions based upon reading the instructions from the computer-readable media. Some non-limiting examples of the disclosure can include (or utilize) a control device such as an automation device, a special purpose or general purpose computer including various computer hardware, software, firmware, and so on, consistent with the discussion below. As specific examples, a control device can include a processor, a microcontroller, a field-programmable gate array, a programmable logic controller, logic gates etc., and other typical components that are known in the art for implementation of appropriate functionality (e.g., memory, communication systems, power sources, user interfaces and other inputs, etc.).

The term “article of manufacture” as used herein is intended to encompass a computer program accessible from any computer-readable device, carrier (e.g., non-transitory signals), or media (e.g., non-transitory media). For example, computer-readable media can include but are not limited to magnetic storage devices (e.g., hard disk, floppy disk, magnetic strips, and so on), optical disks (e.g., compact disk (CD), digital versatile disk (DVD), and so on), smart cards, and flash memory devices (e.g., card, stick, and so on). Additionally it should be appreciated that a carrier wave can be employed to carry computer-readable electronic data such as those used in transmitting and receiving electronic mail or in accessing a network such as the Internet or a local area network (LAN). Those skilled in the art will recognize that many modifications can be made to these configurations without departing from the scope or spirit of the claimed subject matter.

Certain operations of methods according to the disclosure, or of systems executing those methods, can be represented schematically in the FIGS. or otherwise discussed herein. Unless otherwise specified or limited, representation in the FIGS. of particular operations in particular spatial order can not necessarily require those operations to be executed in a particular sequence corresponding to the particular spatial order. Correspondingly, certain operations represented in the FIGS., or otherwise disclosed herein, can be executed in different orders than are expressly illustrated or described, as appropriate for particular non-limiting examples of the disclosure. Further, in some non-limiting examples, certain operations can be executed in parallel, including by dedicated parallel processing devices, or separate computing devices configured to interoperate as part of a large system.

As used herein in the context of computer implementation, unless otherwise specified or limited, the terms “component,” “system,” “module,” and the like are intended to encompass part or all of computer-related systems that include hardware, software, a combination of hardware and software, or software in execution. For example, a component can be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) can reside within a process or thread of execution, can be localized on one computer, can be distributed between two or more computers or other processor devices, or can be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as non-limiting examples of the disclosure, of the utilized features and implemented capabilities of such device or system.

As used herein, unless otherwise defined or limited, ordinal numbers are used herein for convenience of reference based generally on the order in which particular components are presented for the relevant part of the disclosure. In this regard, for example, designations such as “first,” “second,” etc., generally indicate only the order in which the relevant component is introduced for discussion and generally do not indicate or require a particular spatial arrangement, functional or structural primacy or order.

As used herein, unless otherwise defined or limited, directional terms are used for convenience of reference for discussion of particular figures or examples. For example, references to downward (or other) directions or top (or other) positions can be used to discuss aspects of a particular example or figure, but do not necessarily require similar orientation or geometry in all installations or configurations.

This discussion is presented to enable a person skilled in the art to make and use non-limiting examples of the disclosure. Various modifications to the illustrated examples will be readily apparent to those skilled in the art, and the generic principles herein can be applied to other examples and applications without departing from the principles disclosed herein. Thus, non-limiting examples of the disclosure are not intended to be limited to non-limiting examples shown, but are to be accorded the widest scope consistent with the principles and features disclosed herein and the claims below. The following detailed description is to be read with reference to the figures, in which like elements in different figures have like reference numerals. The figures, which are not necessarily to scale, depict selected examples and are not intended to limit the scope of the disclosure. Skilled artisans will recognize the examples provided herein have many useful alternatives and fall within the scope of the disclosure.

Various features and advantages of the disclosure are set forth in the following claims.

We claim:

1. A method of detecting an analyte within a sample, the method comprising:
providing a first electromagnetic radiation to the sample in a manner that resonantly excites mechanical vibrations in the analyte;
providing a second electromagnetic radiation to the sample so as to interact with the vibrating analyte, wherein a third electromagnetic radiation is produced based on the interaction;
receiving the third electromagnetic radiation; and
determining the presence of the analyte based on the received a third electromagnetic radiation.

2. The method of claim 1, wherein the first electromagnetic radiation has a substantially uniform frequency equal to a resonance frequency of the analyte.

3. The method of claim 1, wherein the first electromagnetic radiation is within the microwave range.

4. The method of claim 1, wherein the frequency of the first electromagnetic radiation is swept over a target frequency range in which a resonant frequency of the analyte exists.

5. The method of claim 1, wherein the first electromagnetic radiation has a frequency of more than 1 GHz, more than 3 GHz, more than 5 GHz, more than 8 GHz, less than 100 GHz, less than 50 GHz, less than 15 GHz, between 1 GHz and 100 GHz, between 3 GHz and 50 GHz, or between 5 GHz and 15 GHz.

6. The method of claim 1, wherein the frequency of the third electromagnetic radiation is shifted from the second electromagnetic radiation by an amount equal to the resonance frequency of the analyte.

7. The method of claim 1, further comprising filtering out the third electromagnetic radiation from background electromagnetic radiation using a filter.

8. The method of claim 7, wherein the filter is an optical filter, and wherein filtering out the third electromagnetic radiation from background electromagnetic radiation using the filter occurs before receiving the third electromagnetic radiation.

9. The method of claim 7, wherein the third electromagnetic radiation is passed through a vapor cell that is a reference cell with an absorption line matched to the frequency of the second electromagnetic radiation.

10. The method of claim 1, wherein the second electromagnetic radiation is within the visible light range or the infrared range.

11. The method of claim 1, further comprising:
determining a concentration of the analyte within the sample based on the third electromagnetic radiation.

12. The method of claim 1, wherein at least a part of the providing the first electromagnetic radiation and providing the second electromagnetic radiation occur simultaneously.

13. The method of claim 1, determining the presence of the analyte includes comparing the spectrum of the received a third electromagnetic radiation to previously acquired spectra of a reference sample containing the analyte.

14. The method of claim 1, wherein the analyte is a pathogen.

15. A system for detecting an analyte within a sample, the system comprising:
a first energy source configured to provide a first electromagnetic radiation to the sample in a manner that resonantly excites mechanical vibrations in the analyte;
a second energy source configured to provide a second electromagnetic radiation to the sample so as to interact with vibrating analyte, wherein a third electromagnetic radiation is produced based on the interaction;
a receiver comprising:
a filter configured to separate the third electromagnetic radiation from background electromagnetic radiation; and
a detector configured to receive the filtered a third electromagnetic radiation and produce a detection signal; and
a processor configured to provide information regarding the presence of the analyte based on the detection signal.

16. The system of claim 15, wherein the system is integrated within at least one of:
a heating, ventilation, and air conditioning (HVAC) system; or
an ultraviolet (UV) disinfectant system;
a filtering system;
a fluid supply system that is a water supply system; or
a duct.

17. The system of claim 15, wherein the first energy source is a microwave emitter and the first electromagnetic radiation is within the microwave range, and
wherein the second energy source is a laser emitter and the second electromagnetic radiation is within the visible light range or the infrared range.

18. The system of claim 15, wherein the filter is a vapor cell that is a reference cell with an absorption line matched to the frequency of the second electromagnetic radiation, the filter being positioned in front of the detector.

19. The system of claim 15 further comprising:
a second receiver comprising:
a second filter configured to separate the third electromagnetic radiation from background electromagnetic radiation; and
a second detector configured to receive the filtered a third electromagnetic radiation and produce a detection signal,
wherein the second receiver is positioned on the opposite side of the sample as the second energy source so as to receive transmitted second electromagnetic radiation.

20. A computer-implemented method for determining a resonant frequency of an analyte, the method comprising:
causing, using one or more computing devices, a first energy source to emit first electromagnetic radiation towards a sample that includes the analyte;
causing, using the one or more computing devices, a second energy source to emit second electromagnetic radiation, different from the first electromagnetic radiation, towards the sample that includes the analyte;
receiving, using a detector and the one or more computing devices, third electromagnetic radiation different from the first electromagnetic radiation and the second electromagnetic radiation; and
determining, using the one or more computing devices, a resonant frequency of the analyte based on the received third electromagnetic radiation.
